(19) United States
(12) Patent Application Publication
(10) Pub. No.: US 2010/0317599 A1
(43) Pub. Date: Dec. 16, 2010

(54) PROCESS FOR THE PRODUCTION OF A PEPTIDE

(76) Inventors: Alrik Pieter Los, Dijkstraat (NL); Jan Metske Laan Van Der, Breda (NL); Cornelis Maria Jacobus Sagt, Utrecht (NL); Herman Jan Pel, Terschuur (NL)

Correspondence Address: NIXON & VANDERHYE, PC, 901 NORTH GLEBE ROAD, 11TH FLOOR, ARLINGTON, VA, 22203, US

(21) Appl. No.: 12/867,661

(22) PCT Filed: Jan. 29, 2009
(86) PCT No.: PCT/EP2009/051023

§ 371 Date: Aug. 13, 2010

(30) Foreign Application Priority Data
Feb. 13, 2008 (EP) 08101579.4
Jul. 22, 2008 (EP) 08160877.0

(51) Int. Cl. A61K 38/16; C07K 14/00; C07H 21/04; C12N 15/63; C12N 1/15; C12P 21/02; A61P 43/00; A23J 3/00

(52) U.S. Cl. 514/21.2; 530/350; 536/23.1; 435/320.1; 435/254.11; 536/25.3; 435/69.1; 426/656

(57) ABSTRACT

The present invention describes a polypeptide, comprising at least one repeat domain, further comprising a recombinant peptide of interest. The present invention further describes a process for the production of a recombinant peptide of interest. The invention further describes the use of a repeat protein for the production of a recombinant peptide of interest.

### FIELD OF THE INVENTION

The present invention relates to the production of a peptide.

### BACKGROUND OF THE INVENTION

The production of peptides is known from the prior art. Current production of peptides is performed in various ways.

The state of the art process for the production of peptides is by means of fermentation, wherein multimeric genes encoding tandem repeats of the peptide are fused to a polynucleotide encoding a carrier protein. The fusion protein is produced by fermentation in a host cell, e.g. E. coli. Subsequently, the fusion protein is isolated and the peptides are cleaved from the carrier protein (Metlitskaia et al., Recombinant antimicrobial peptides efficiently produced using novel cloning and purification processes. Biotechnol. Appl. Biochem. 2004, 39:339-345). This process has the disadvantage that the yield is generally low, probably due to the biased amino acid composition of peptide multimers causing problems with protein folding and amino acid/tRNA pool limitations. In addition, the fusion protein generally accumulates in inclusion bodies. Isolation of the fusion protein from the inclusion bodies is laborious and cumbersome.

Consequently, there is a need for alternative processes for the fermentative production of peptides. It is therefore an object of the present invention to provide such alternative processes.

### DESCRIPTION OF THE FIGURES

FIG. 1. Schematic representation of a repeat protein, a repeat domain, a repeat unit and the positions of a carrier, tag, and variable loops with and without recombinant peptides.

In (A), the variable loop is located on the C-terminal side of the repeat units.
In (B), the variable loop is located within the repeat units.
In (C), a repeat domain is depicted, comprised of several repeat units.

FIG. 2. Schematic examples of repeat proteins comprising recombinant peptides. Amino acid motifs flanking the peptides are represented by black filled squares; peptides are represented by open squares comprising the term ‘pep’.

(A) depicts a single peptide, flanked by amino acid motifs facilitating e.g. isolation, in each of the variable loops on the C-terminal side of the repeat units.
(B) depicts two peptides, flanked by amino acid motifs facilitating e.g. isolation, in some of the variable loops on the C-terminal side of the repeat units.
(C) depicts three consecutive peptides in some of the variable loops on the C-terminal side of the repeat units.

FIG. 3. Map of pGBE01Ank1 for expression of His-tagged Ankyrin repeat protein in E. coli. pGBE01Ank1 is a pMS470-based plasmid comprising the tac promoter. Depicted is the His-tagged Ankyrin repeat protein cloned into the HindIII/NdeI sites. The tac promoter is used to express the His-tagged Ankyrin repeat protein. pGBE01Ank1 is representative for pGBE01Ank2, which comprises His-tagged Ankyrin repeat protein and recombinant peptide IPP.

FIG. 4. Detection of Ankyrin repeat protein and Ankyrin repeat protein comprising recombinant peptide IPP, expressed in E. coli.

(4A): SDS-PAGE detection of Ankyrin repeat protein and Ankyrin repeat protein comprising recombinant peptide IPP, expressed in E. coli. Lane 1 contains a molecular weight marker; lane 2 contains E.coli RV308-Ank1 (Ankyrin) lysate; lane 2 contains E.coli RV308-Ank2 (Ankyrin+IPP) lysate.
(4B): Western blot detection of Ankyrin repeat protein and Ankyrin repeat protein comprising recombinant peptide IPP, expressed in E. coli, using a His-tag specific antibody. Lane 1 contains a molecular weight marker; lane 2 contains E.coli RV308-Ank1 (Ankyrin) lysate; lane 2 contains E.coli RV308-Ank2 (Ankyrin+IPP) lysate.

FIG. 5. Map of pGBFINAnk1 for expression of chimeric glucoamylase-Ankyrin repeat protein comprising recombinant peptide IPP in A. niger. pGBFINAnk1 is a pGBFIN5-based plasmid. Depicted are the glucoamylase fragment (glaA) and the Ankyrin repeat protein comprising recombinant peptide IPP (Ank+IPP+FLAG) expressed from the glucoamylase promoter (PglaA). In addition, the selection marker gene (amdS), expressed from the gpdA promoter (PgpdA) and the glucoamylase flanks (3′glaA and 3″glaA) of the expression cassette are depicted.

### DETAILED DESCRIPTION OF THE INVENTION

Surprisingly, it has been established that production of a recombinant peptide of interest can be accomplished by production of a recombinant repeat protein, wherein the repeat protein comprises a recombinant peptide of interest.

Therefore, in a first aspect of the present invention there is provided a polypeptide, comprising at least one repeat domain, further comprising a recombinant peptide of interest.

In the context of the present invention the terms “polypeptide” and “protein” are identical and throughout the description of the present invention can be read interchangeably. Consequently, a “repeat protein” and a “polypeptide comprising at least one repeat domain” are identical terms.

The methods of the prior art using fermentative production of peptides, as reflected by (Metlitskaia et al., Recombinant antimicrobial peptides efficiently produced using novel cloning and purification processes. Biotechnol. Appl. Biochem. 2004, 39:339-345) do not use a repeat protein for the production of a peptide of interest. In alternative methods for production of peptides, production is performed by chemical synthesis, wherein the amino acids are coupled by chemical processes (for review see: Synthetic peptides, A User's guide, edited by Gregory A. Grant, 1992, W.H. Freeman and Company, New York, ISBN 0-7167-7009-1). This way of synthesis has the disadvantage that it is laborious and therefore expensive; the process is getting progressively laborious with the length of the peptide chain. Furthermore, with increasing length of the synthesized peptide, chances on the occurrence of unwanted side-reactions increase, thus decreasing the yield progressively with the length of the peptide.

In the context of the present invention, the term “recombinant” refers to any genetic modification not exclusively involving naturally occurring processes and/or genetic modifications induced by subjecting the host cell to random mutagenesis. Consequently, combinations of recombinant and naturally occurring processes and/or genetic modifications induced by subjecting the host cell to random mutagenesis are construed as being recombinant. Preferably, recombinant genetic modification does not involve naturally occurring processes and/or genetic modifications induced by subjecting the host cell to random mutagenesis

A “peptide” or “oligopeptide” is herein referred to as a molecule comprised of at least two amino acids arranged in a linear chain and joined together by peptide bonds between the carboxyl and amino groups of adjacent amino acid residues. The terms “peptide” and “oligopeptide” are considered synonymous (as is commonly recognized) and each term can be used interchangeably as the context requires. A “polypeptide” is herein referred to as a molecule comprising at least 40 amino acids.

The term “a peptide” is synonymous with the term “at least one peptide”.

The polypeptide according to the invention comprises at least one repeat domain, preferably the polypeptide comprises up to four repeat domains, more preferably the polypeptide comprises up to two repeat domains, most preferably the polypeptide comprises one repeat domain.

In the context of the present invention, the term “repeat domain” refers to a protein domain comprising two or more, preferably consecutive, repeat units as structural units, wherein said structural units have the same fold, and stack tightly to create a superhelical structure having a joint hydrophobic core (reviewed in Kobe and Kajava, When protein folding is simplified to protein coiling: the continuum of solenoid protein structures. Trends Biochem. Sci. 2000, 25:509-515). The term “structural unit” refers to a locally ordered part of a polypeptide, formed by three-dimensional interactions between two or more segments of secondary structure that are near one another along the polypeptide chain.

In the context of the present invention, the term “repeat unit” refers to amino acid sequences comprising one or more sequence motif, designated herein as “repeat motif”, which exhibit a defined folding topology common to all said sequence motifs determining the fold of the protein (Kajava, What Curves alpha-Solenoids? Evidence for an alpha-helical toroid structure of Rpn1 and Rpn2 proteins of the 26 S proteasome. J. Biol. Chem. 2002, 277:49791-49798). The term “folding topology” refers to the tertiary structure of said repeat units. The folding topology will be determined by structural elements forming at least parts of alpha-helices or beta-strands, or amino acid stretches forming linear polypeptides or loops, or any combination of alpha-helices, beta-strands and/or linear polypeptides/loops.

In an embodiment of the present invention, the repeat motif of the repeat unit occurs in one or more naturally occurring proteins, in which said repeat units are present. In another embodiment, the sequence motif of the repeat unit does not occur in nature and is a designed motif or a variant of a naturally occurring motif.

The repeat unit may or may not comprise a variable loop. A repeat unit comprising a variable loop, is thus comprised of one or more repeat motif and one or more variable loop, wherein said repeat motif comprises at least one structural element.

The term “variable loop” is herein referred to as a stretch of amino acids that connects the most C-terminal side structural element from a repeat motif with the first N-terminal side structural element of the adjacent repeat motif at the C-terminal side of the repeat domain.

When a repeat unit comprises a variable loop, the variable loop is preferably located on the C-terminal side of the repeat units.

An example of a repeat domain comprising two or more consecutive repeat units comprising said loops has the structure of “repeat motif—loop—repeat motif—loop—repeat motif etc”, wherein a “repeat motif—loop” forms one repeat unit.

Alternatively, the repeat unit comprises a variable loop, which is located within the repeat unit. Said loop is herein referred to as a stretch of amino acids that connect two adjacent structural elements within the repeat unit.

A repeat unit according to the invention may have any size. Preferably, the repeat unit comprises between 10 and 60 amino acids, more preferably, between 20 and 40 amino acids, even more preferably between 32 and 35 amino acids, even more preferably between 32 and 34 amino acids and most preferably the repeat unit comprises 33 amino acids.

A repeat domain according to the invention comprises two or more repeat units. Preferably, the repeat domain comprises up to 60 repeat units, even more preferably up to 30 repeat units, even more preferably up to 15 repeat units, even more preferably up to 6 repeat units. Even more preferably, the repeat domain comprises 2, 3, 4, 5, or 6 repeat units, even more preferably 4, 5, or 6 repeat units and most preferably 5 repeat units.

The polypeptide according to the invention optionally comprises additional domains that are no repeat domains. Examples of domains that are no repeat domains are, but should not be construed as limitations of the present invention, peptide and/or polypeptide tags. These sequences are well-known and available to the person skilled in the art. The term “peptide tag or polypeptide tag” refers to an amino acid sequence attached to a polypeptide, where said amino acid sequence is usable for e.g. the purification, detection, or targeting of said polypeptide.

The polypeptide according to the invention optionally comprises a C-terminal and or N-terminal capping unit. A capping unit is herein referred to as a polypeptide unit that is located at the C or N-terminus of a polypeptide or repeat domain. Capping units preferably are more hydrophilic than the (hydrophobic) rest of the protein and consequently shield the hydrophobic part to increase solubility (Kohl et al., Designed to be stable: crystal structure of a consensus ankyrin repeat protein. 2003, Proc Natl Acad Sci USA. 100:1700-1705).

Preferably, the polypeptide according to the invention is derived from a naturally occurring parental polypeptide. More preferably, the naturally occurring polypeptide is a known repeat protein. For specific uses of the polypeptide according to the invention or the recombinant peptide of interest according to the invention, the selection of the repeat protein may be made according to such use. Where e.g. the repeat protein comprising the recombinant peptide of interest or the recombinant peptide of interest according to the invention is to be used in food applications, a repeat protein may be selected from a food-grade organism such as Saccharomyces cerevisiae. Specific uses of the polypeptide according to the invention include, but are not limited to, food, (animal) feed, pharmaceutical, agricultural such as crop-protection, and/or personal care applications.

Accordingly, compositions for use in food-, (animal) feed-, pharmaceutical-, agricultural- such as crop-protection, and/or personal care applications, comprising a for the purpose acceptable helper compound and a polypeptide according to the invention are provided by the present invention. Furthermore, the polypeptide according to the invention for use as a medicament is provided by the invention.

The repeat protein may be any repeat protein. Repeat proteins are known to the skilled person and these may conveniently applied for use in the invention. Repeat proteins are reviewed by Main E R et al. (Main et al., The folding and design of repeat proteins: reaching a consensus. Curr Opin Struct Biol. 2003 13:482-9. Review); the design of repeat proteins is inter alia described in Binz et al., Designing repeat proteins: well-expressed, soluble and stable proteins from combinatorial libraries of consensus ankyrin repeat proteins. 2003, J Mol Biol. 332:489-503; Kohl et al., Designed to be stable: crystal structure of a consensus ankyrin repeat protein. 2003, Proc Natl Acad Sci USA. 100:1700-1705. Repeat proteins can be identified in organisms by making use of PFAM motifs (Finn et al., Pfam: clans, web tools and services. 2006, Nucleic Acids Res. Database Issue 34:D247-D251). Each repeat protein has a specific PFAM motif based on the consensus sequence of the repeat protein. In WO02/20565, recombinant modulation of repeat proteins is described to construct recombinant binding proteins. No production of a peptide of interest is described. However, the general knowledge on repeat proteins and the techniques used for the modulation of the repeat proteins of WO02/20565 can conveniently be used in the invention and WO02/20565 is herein incorporated by reference.

Preferred repeat proteins are selected from the list of: ankyrin repeat protein (Kalus et al., NMR structural characterization of the CDK inhibitor p19INK4d, 1997, FEBS Lett. 401:127-132), Leucine-rich repeat protein (Kobe et al., Crystal structure of porcine ribonuclease inhibitor, a protein with leucine-rich repeats. 1993, Nature 366:751-756), WD40 or WD40-like or WD domain/G-beta repeat protein (Neer et al., The ancient regulatory-protein family of WD-repeat proteins. 1994, Nature 371:297-300, HEAT repeat (Groves et al., The structure of the protein phosphatase 2A PR65/A subunit reveals the conformation of its 15 tandemly repeated HEAT motifs. 1999, Cell. 96:99-110, PPR repeat protein (Small and Peeters, The PPR motif—a TPR-related motif prevalent in plant organellar proteins. 2000, Trends Biochem Sci 25:45-47), Tetratricopeptide repeat protein (Pallen et al., Tetratricopeptide-like repeats in type-III-secretion chaperones and regulators. 2003, FEMS Microbiol Lett 223:53-60; Blatch and Lässle. The tetratricopeptide repeat: a structural motif mediating protein-protein interactions. 1999, Bioessays. 21:932-939; Lamb et al., Tetratrico peptide repeat interactions: to TPR or not to TPR? 1995, Trends Biochem Sci 20:257-259), bacterial transferase hexapeptide (Raetz and Roderick. A left-handed parallel beta helix in the structure of UDP-N-acetylglucosamine acyltransferase. 1995, Science 270:997-1000), RPEL repeat protein (Favot et al., Overexpression of a family of RPEL proteins modifies cell shape. 2005, FEBS Lett. 579:100-104, Armadillo/beta-catenin-like repeat protein (Huber et al., Three-dimensional structure of the armadillo repeat region of beta-catenin, 1997, Cell 90:871-882), PQ loop repeat protein (Cherqui et al., The targeting of cystinosin to the lysosomal membrane requires a tyrosine-based signal and a novel sorting motif. 2001, J Biol Chem 276:13314-13321).

More preferred repeat proteins are selected from the list of: ankyrin repeat protein, Leucine-rich repeat protein, WD40 or WD40-like or WD domain/G-beta repeat protein, HEAT repeat protein, bacterial transferase hexapeptide, RPEL repeat protein, PQ loop repeat.

Even more preferred repeat proteins are selected from the list of: ankyrin repeat protein, WD40 or WD40-like or WD domain/G-beta repeat protein, HEAT repeat protein, RPEL repeat protein, PQ loop repeat protein.

Even more preferred repeat proteins are selected from the list of: ankyrin repeat protein, WD40 or WD40-like or WD domain/G-beta repeat protein HEAT repeat protein.

Even more preferred proteins are selected from the list of: ankyrin repeat protein and HEAT repeat protein.

Most preferred repeat proteins are ankyrin repeat proteins. Examples of ankyrin repeat proteins are the ankyrin repeat proteins of Saccharomyces cerevisiae and of A.niger CBS 513.88 (Pel et al., Genome sequencing and analysis of the versatile cell factory Aspergillus niger CBS 513.88. 2007, Nat Biotechnol. 25:189-90), e.g. An11g08920 or An18g00540.

According to the invention, the polypeptide according to the invention or parental repeat protein may be native or foreign to the host cell wherein the polypeptide is produced or to the organism of application. A native polypeptide generally has the advantage that native proteins usually are more stable and less susceptible to degradation in their native environment. The polypeptide according to the invention or parental repeat protein may comprise designed modifications.

The polypeptide according to the invention may be a chimeric polypeptide, being comprised of two or more parts of repeat proteins. Said parts may be obtained from distinct proteins, said distinct proteins may be derived from different host cell genus or species.

When the polypeptide according to the invention is to be secreted from the host cell into the cultivation medium, an appropriate signal sequence can be added to the polypeptide in order to direct the de novo synthesized polypeptide to the secretion route of the host cell. The person skilled in the art knows to select an appropriate signal sequence for a specific host. The signal sequence may be native to the host cell, or may be foreign to the host cell. As an example, a signal sequence from a protein native to the host cell can be used. Preferably, said native protein is a highly secreted protein, i.e. a protein that is secreted in amounts higher than 10% of the total amount of protein being secreted.

As an alternative for a signal sequence, the polypeptide of the invention can be fused to a secreted carrier protein, or part thereof. Such chimeric construct is directed to the secretion route by means of the signal sequence of the carrier protein, or part thereof. In addition, the carrier protein will provide a stabilizing effect to the polypeptide according to the invention and or may enhance solubility. Such carrier protein may be any protein. Preferably, a highly secreted protein is used as a carrier protein. The carrier protein may be native or foreign to the polypeptide according to the invention. The carrier protein may be native of may be foreign to the host cell. Examples of such carrier proteins are glucoamylase, prepro sequence of alpha-Mating factor, cellulose binding domain of Clostridium cellulovorans cellulose binding protein A, glutathione S-transferase, chitin binding domain of Bacillus circulans chitinase A1, maltose binding domain encoded by the malE gene of E. coli K12, beta-galactosidase, and alkaline phosphatase. A preferred carrier protein for expression of such chimeric construct in Aspergillus cells is glucoamylase. The carrier protein and polypeptide according to the invention may contain a specific amino acid motif to facilitate isolation of the polypeptide; the polypeptide according to the invention may be released by a special releasing agent. The releasing agent may be a proteolytic enzyme or a chemical agent. An example of such amino acid motif is the KEX protease cleavage site, which is well-known to the person skilled in the art.

In the context of the present invention, the terms “peptide of interest” and “recombinant peptide of interest” refer to a peptide comprising a trait of interest. Said trait of interest may be any trait under consideration that is identified or identifiable.

The trait of interest may be on the field of food, (animal) feed, pharmaceutical, agricultural such as crop-protection, and/or personal care applications. An example of such peptide for food use is IPP for lowering blood pressure levels, or poly IPP after cleavage with a proline specific endoprotease, as described in WO2006/089921. An example of such peptide for pharmaceutical use is the Gila Monster (Heloderma suspectum) peptide exenatide (available as Byetta™ from Byetta, US) as a medicament for diabetes II. The amino acid sequence of exenatide is His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser. An example of a personal care peptide is the (Palmitoyl)-Lys-Thr-Thr-Lys-Ser pentapeptide described in US 20070237735.

Other examples of (identified) peptides of interest are IPP and VPP (Nakamura et al., Purification and Characterization of Angiotensin I-Converting Enzyme Inhibitors from Sour Milk. 1995, J. Dairy Sci. 78:777-783). Preferably, said trait of interest is a biological activity.

The recombinant peptide of interest according to the invention may be any recombinant peptide. Preferably, said peptide is a peptide comprising biological activity. A peptide comprising biological activity is defined as a peptide that by virtue of interaction with an organism, microorganism, or part(s) thereof, has a direct or indirect effect on that organism, microorganism, or part(s) thereof. Such effect can e.g. be a stimulating, modulating, inducing or inhibiting effect. Biological activity of a peptide includes peptide hormone activity and receptor binding activity. More preferably, said peptide binds to or interacts with a receptor or protein, and stimulates or inhibits receptor or protein activity, or directly or indirectly modifies the protein or receptor. Alternatively, said peptide prevents activation, inhibition or modification of the receptor or protein by another molecule.

Preferably, the recombinant peptide according to the invention is not an enzyme.

The recombinant peptide of interest according to the invention may e.g. be a peptide that has directly or indirectly effect on a metabolic syndrome, e.g. modulating cholesterol levels, and/or modulating, preferably lowering blood pressure levels, and/or modulating insulin levels, and/or dipeptidylpeptidase IV (DPP IV; EC 3.4.14.5), and/or mood, and/or satiety, and/or diseases temporally mend in connection with metabolic diseases and/or biological activity relevant for personal care applications.

The recombinant peptide of interest according to the invention may have any size. Preferably, the recombinant peptide comprises up to 1500 amino acids, more preferably up to 1000 amino acids, even more preferably up to 800 amino acids, even more preferably up to 700 amino acids, even more preferably up to 600 amino acids, even more preferably up to 500 amino acids, even more preferably up to 400 amino acids, even more preferably up to 300 amino acids, even more preferably up to 250 amino acids, even more preferably up to 200 amino acids, even more preferably up to 150 amino acids, even more preferably up to 140 amino acids, even more preferably up to 130 amino acids, even more preferably up to 120 amino acids, even more preferably up to 110 amino acids, even more preferably up to 100 amino acids, even more preferably up to 90 amino acids, even more preferably up to 80 amino acids, even more preferably up to 70 amino acids, even more preferably 60 amino acids, even more preferably up to 50 amino acids, even more preferably up to 40 amino acids, even more preferably up to 30 amino acids, even more preferably up to 25 amino acids, even more preferably up to 20 amino acids, even more preferably up to 16 amino acids, even more preferably up to 10 amino acids, even more preferably up to 6 amino acids, even more preferably between 2 and 6 amino acids, and most preferably the recombinant peptide is a tripeptide. According to an embodiment of the invention, the recombinant peptide comprises between 30 and 60 amino acids. According to another embodiment, the recombinant peptide comprises between 16 and 30 amino acids. According to yet another embodiment, the recombinant peptide comprises between 6 and 15 amino acids. Preferred peptides are hypotensive peptides such as LPP, IPP, VPP; and DPP-IV inhibiting peptides such as VPI, VPV, IPV, LPI, LPV, YPI, YPV, FPI, FPV, PPI, PPV, RPI, RPV, QPI, QPV, TPI and TPV. Further examples of preferred peptides are DPP, LPP, IPP, VPP, MAP, VPI, XPX and XPPX, wherein “X” may be any natural or non-natural amino acid.

The recombinant peptide according to the invention may be a recombinant version of a peptide occurring natural in nature. The recombinant peptide may be a designed peptide, not occurring naturally in nature.

According to the invention, the recombinant peptide may comprise naturally occurring amino acids, but may also comprise non-natural amino acids. A non-natural amino acid is herein referred to as an amino acid not being a natural amino acid that can be incorporated into a peptide or polypeptide. Examples of non-natural amino acids are beta-amino acids and modified natural amino acids that can still be incorporated into a peptide or polypeptide. (Daniels et al., 2007, High-resolution structure of a beta-peptide bundle. J Am Chem Soc. 129:1532-3.; Hendrickson et al., 2004, Incorporation of nonnatural amino acids into proteins. Annu Rev Biochem. 73:147-76). Recombinant peptides comprising mixtures of natural and non-natural amino acids are explicitly comprised in the invention.

Preferably, the recombinant peptide according to the invention is comprised within a repeat domain of the polypeptide according to the invention. More preferably, the recombinant peptide is comprised in a repeat unit. Even more preferably, the recombinant peptide is comprised in the variable loop between two repeat motifs.

According to the invention, multiple recombinant peptides may be present in the polypeptide of the invention, preferably as a multimer within a single repeat unit of the repeat protein. The recombinant peptides may be present as multimers in one or more repeat units. According to the invention, distinct recombinant peptides may be present in the polypeptide according to the invention.

Preferably, the recombinant peptide according to the invention is present in a repeat unit as a multimer of 2, 3, 4, 5, 6, 7, 8, 9, 10, 11, 12, 13, or more. More preferably, the recombinant peptide is present in a repeat unit as a multimer whereby the total amount of amino acids of such multimer is up to 50 amino acids.

Optionally, the recombinant peptide according to the invention is flanked on the C-terminal and/or the N-terminal side by an amino acid motif that facilitates identification, isolation and/or purification of the recombinant peptide and/or that stabilizes the repeat protein. Such amino acid motif may be cellulose binding domain, glutathione S-transferase, chitin binding, domain, maltose binding domain, β-galactosidase, alkaline phosphatase, polyarginine-tag, polyhistidine-tag, FLAG-tag, myc-tag, VSV-tag, HA-tag, and Protein A.

Such amino acid motif may also be a sequence that facilitates stabilisation of the repeat protein or expression of the repeat protein. For example, the amino acid motif may be required for the correct folding of the variable loop and/or entire repeat protein, or the amino acid motif may compensate for negative or positive charges within the recombinant peptide(s).

Such amino acid motif may be a cleaving site facilitating release of the recombinant peptide from the polypeptide according to the invention by proteolysis.

Release of the recombinant peptide according to the invention may be performed by any process known to the skilled person. For instance, the recombinant peptide(s) may be released by digestion of the polypeptide of the invention with at least one appropriate proteolytic enzyme. Examples of proteolytic enzymes are inter alia described in http://www.expasy.ch/tools/peptidecutter/. An example in which a peptide is isolated after proteolytic cleavage of a protein is described in WO2006/089921, wherein tripeptide IPP is released from casein by proteolytic cleavage of casein by a prolyl endoprotease.

Optionally, the recombinant peptide according to the invention may subsequently be isolated. The isolation may result in isolation of the recombinant peptide of interest per se. The isolation may also result in the isolation of a peptide fragment comprising the recombinant peptide of interest.

Specific uses of the recombinant peptide according to the invention and/or peptide fragment comprising the recombinant peptide of interest include, but are not limited to, food, (animal) feed, pharmaceutical, agricultural such as crop-protection, and/or personal care applications. Examples of peptides used in these applications are those described earlier herein.

Accordingly, compositions for use in food-, (animal) feed-, pharmaceutical-, agricultural- such as crop-protection, and/or personal care applications, comprising a for the purpose acceptable helper compound and a recombinant peptide according to the invention or a peptide fragment comprising the recombinant peptide according to the invention are provided by the present invention. Furthermore, the recombinant peptide according to the invention and/or a peptide fragment comprising the recombinant peptide according to the invention for use as a medicament is provided by the invention.

A method for releasing peptides from polypeptides wherein a peptide naturally occurs may be used for the release of the recombinant peptide of the invention. Such method is known from the prior art for the production of peptides involving the hydrolysis of polypeptides or polypeptide mixtures wherein a peptide of interest is present, whereby the peptide of interest is released from the polypeptide. For example, the anti-hypertensive peptide XPP, wherein “X” may be any natural amino acid, is released from casein after processing with an Aspergillus oryzae protease (Mizuno et al., Release of Short and Proline-Rich Antihypertensive Peptides from Casein Hydrolysate with an Aspergillus oryzae Protease. 2004, J. Dairy Sci. 87:3183-3188). As such, the method described by Mizuno for the production of a peptide has the disadvantage that the peptide is contaminated with a large amount of other peptides and polypeptide fragments, due to the unfavourable ratio between peptide of interest versus polypeptide. In fact, if the total of IPP, VPP and LPP peptides present in casein would be isolated from casein, maximally 16.24 mg Antihypertensive peptide (i.e. IPP, VPP and LPP) would be isolated per gram of casein. In contrast, if according to the invention 4 recombinant IPP peptides are inserted into an ankyrin repeat protein of 166 amino acids, a yield of 67 mg Antihypertensive peptide (i.e. IPP) is isolated per gram of protein. Insertion of more than 4 tripeptides further increases the yield. The present invention thus conveniently allows shifting the ratio of peptide versus polypeptide to a more favourable one by comprising a large number of recombinant peptides into the polypeptide according to the invention.

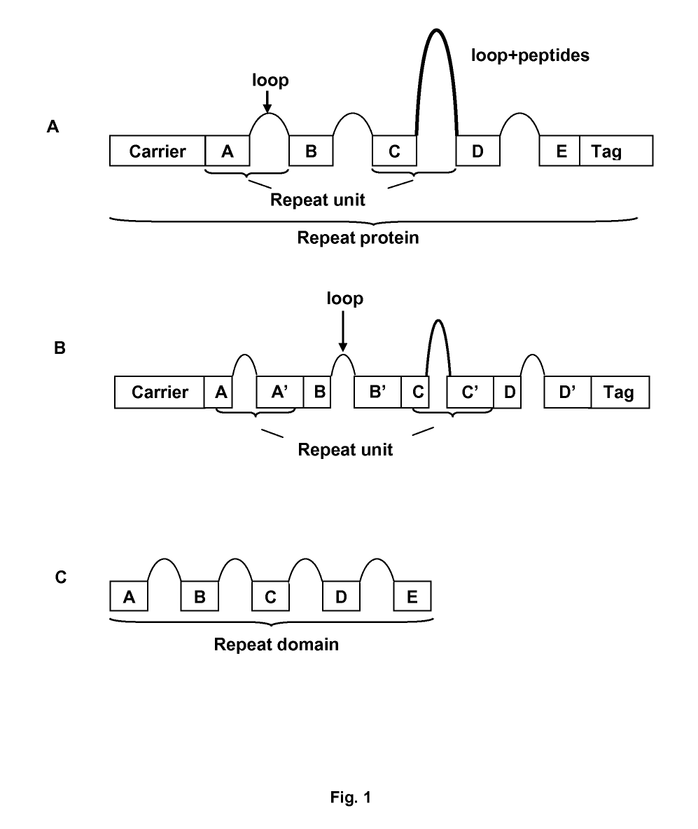

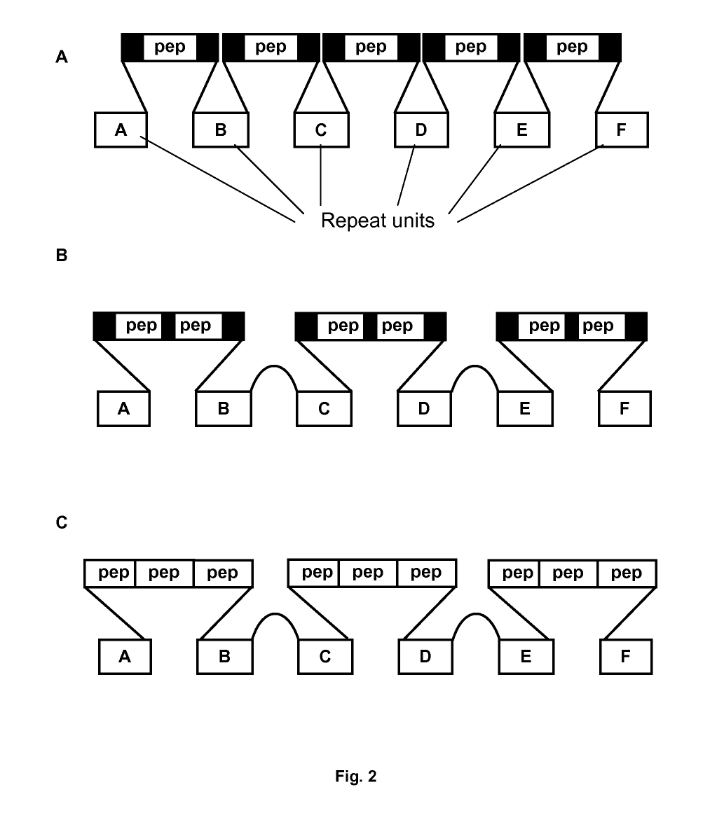

Various make-ups of the polypeptide according to the invention are envisaged as embodiments of the invention. Examples of possible make-ups, which are not to be construed as limitations of the invention, are depicted in FIGS. 1 and 2.

FIG. 1 depicts schematic representations of repeat proteins comprising a repeat domain, a repeat unit, a carrier, a tag, and variable loops with and without peptides.

In (A), the variable loop is located on the C-terminal side of the repeat units.
In (B), the variable loop is located within the repeat units.
In (C), a repeat domain is depicted, comprised of several repeat units.

In FIG. 2, three variants of an ankyrin repeat protein comprising 6 repeat units and peptides are depicted. Amino acid motifs flanking the peptides are represented by black filled squares; peptides are represented by open squares comprising the term ‘pep’.

(A) depicts single peptides, flanked by amino acid motifs facilitating e.g. isolation, in each of the variable loops on the C-terminal side of the repeat units.
(B) depicts two peptides, flanked by amino acid motifs facilitating e.g. isolation, in some of the variable loops on the C-terminal side of the repeat units.
(C) depicts three consecutive peptides in some of the variable loops on the C-terminal side of the repeat units.

In a second aspect of the present invention, there is provided a polynucleotide encoding the polypeptide according to the invention comprising at least one repeat domain, further comprising a recombinant peptide of interest.

Preferably, the recombinant peptide according to the invention is comprised in a repeat domain. More preferably, the recombinant peptide is comprised in a repeat unit. Even more preferably, the recombinant peptide is comprised in the variable loop between two repeat motifs.

According to the invention, distinct recombinant peptides may be present in the polypeptide of the invention.

The term “polynucleotide” is identical to the term “nucleic acid molecule” and can herein be read interchangeably. The term refers to a polynucleotide molecule, which is a ribonucleic acid (RNA) or deoxyribonucleic acid (DNA) molecule, either single stranded or double stranded. A polynucleotide may either be present in isolated form, or be comprised in recombinant nucleic acid molecules or vectors, or be comprised in a host cell.

The polynucleotide according to the invention may be derived from a naturally occurring polynucleotide encoding a repeat protein. Such polynucleotide may have a significant number of mutations, substitutions, insertions and/or deletions when compared to the naturally occurring polynucleotide sequence encoding the repeat protein, while still substantially retaining the repeat units.

The polynucleotide according to the invention may be a synthetic polynucleotide. The synthetic polynucleotide may be optimized in its codon use, preferably according to the methods described in WO2006/077258 and/or PCT/EP2007/055943, which are herein incorporated by reference. PCT/EP2007/055943 addresses codon-pair optimization. Codon-pair optimisation is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence.

In a third aspect of the present invention there is provided a nucleic acid construct comprising the polynucleotide according to the invention.

The term “nucleic acid construct” is herein referred to as a nucleic acid molecule, either single-or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” when the nucleic acid construct contains all the control sequences required for expression of a coding sequence, wherein said control sequences are operably linked to said coding sequence. The term “coding sequence” as defined herein is a sequence, which is transcribed into mRNA and translated into a polypeptide according to the invention. The boundaries of the coding sequence are generally determined by the ATG or other start codon at the 5′-end of the mRNA and a translation stop codon sequence terminating the open reading frame at the 3′-end of the mRNA. A coding sequence can include, but is not limited to, DNA, cDNA, and recombinant nucleic acid sequences.

The term “control sequences” is defined herein to include all components, which are necessary or advantageous for the expression of mRNA and/or a polypeptide, either in vitro or in a host cell. Each control sequence may be native or foreign to the nucleic acid sequence encoding the polypeptide. Such control sequences include, but are not limited to, a leader, Shine-Delgarno sequence, optimal translation initiation sequences (as described in Kozak, 1991, J. Biol. Chem. 266:19867-19870), a polyadenylation sequence, a pro-peptide sequence, a pre-pro-peptide sequence, a promoter, a signal sequence, and a transcription terminator. At a minimum, the control sequences include a promoter, and transcriptional and translational stop signals. Control sequences may be optimized to their specific purpose. Preferred optimized control sequences used in the present invention are those described in WO2006/077258, which is herein incorporated by reference.

The control sequences may be provided with linkers for the purpose of introducing specific restriction sites facilitating ligation of the control sequences with the coding region of the nucleic acid sequence encoding a polypeptide. The term “operably linked” is defined herein as a configuration in which a control sequence is appropriately placed at a position relative to the coding sequence of the DNA sequence such that the control sequence directs the production of a polypeptide.

The control sequence may be an appropriate promoter sequence, a nucleic acid sequence, which is recognized by a host cell for expression of the nucleic acid sequence. The promoter sequence contains transcriptional control sequences, which mediate the expression of the polypeptide. The promoter may be any nucleic acid sequence, which shows transcriptional activity in the cell including mutant, truncated, and hybrid promoters, and may be obtained from genes encoding extracellular or intracellular polypeptides either homologous or heterologous to the cell.

The control sequence may also be a suitable transcription terminator sequence, a sequence recognized by a filamentous fungal cell to terminate transcription. The terminator sequence is operably linked to the 3′-terminus of the nucleic acid sequence encoding the polypeptide. Any terminator, which is functional in the cell, may be used in the present invention.

Preferred terminators for filamentous fungal cells are obtained from the genes encoding A. oryzae TAKA amylase, A. niger glucoamylase (glaA), A. nidulans anthranilate synthase, A. niger alpha-glucosidase, trpC gene and Fusarium oxysporum trypsin-like protease.

The control sequence may also be a suitable leader sequence, a non-translated region of an mRNA which is important for translation by the filamentous fungal cell. The leader sequence is operably linked to the 5′-terminus of the nucleic acid sequence encoding the polypeptide. Any leader sequence, which is functional in the cell, may be used in the present invention.

Preferred leaders for filamentous fungal cells are obtained from the genes encoding A. oryzae TAKA amylase and A. nidulans triose phosphate isomerase and A. niger glaA and phytase.

Other control sequences may be isolated from the Penicillium IPNS gene, or pcbC gene, the beta tubulin gene. All the control sequences cited in WO 01/21779 are herewith incorporated by reference.

The control sequence may also be a polyadenylation sequence, a sequence which is operably linked to the 3′-terminus of the nucleic acid sequence and which, when transcribed, is recognized by the filamentous fungal cell as a signal to add polyadenosine residues to transcribed mRNA. Any polyadenylation sequence, which is functional in the cell, may be used in the present invention.

Preferred polyadenylation sequences for filamentous fungal cells are obtained from the genes encoding A. oryzae TAKA amylase, A. niger glucoamylase, A. nidulans anthranilate synthase, Fusarium oxysporum trypsin-like protease and A. niger alpha-glucosidase.

The term “promoter” is defined herein as a DNA sequence that binds RNA polymerase and directs the polymerase to the correct downstream transcriptional start site of a nucleic acid sequence encoding a biological compound to initiate transcription. RNA polymerase effectively catalyzes the assembly of messenger RNA complementary to the appropriate DNA strand of a coding region. The term “promoter” will also be understood to include the 5′-non-coding region (between promoter and translation start) for translation after transcription into mRNA, cis-acting transcription control elements such as enhancers, and other nucleotide sequences capable of interacting with transcription factors. The promoter may be any appropriate promoter sequence suitable for a eukaryotic or prokaryotic host cell, which shows transcriptional activity, including mutant, truncated, and hybrid promoters, and may be obtained from genes encoding extra-cellular or intracellular polypeptides either homologous (native) or heterologous (foreign) to the cell. The promoter may be a constitutive or inducible promoter. Examples of inducible promoters that can be used are a starch-, copper-, oleic acid-inducible promoters. The promoter may be selected from the group, which includes but is not limited to promoters obtained from the genes encoding A. oryzae TAKA amylase, Rhizomucor miehei aspartic proteinase, A. niger neutral alpha-amylase, A. niger acid stable alpha-amylase, A. niger or A. awamori glucoamylase (glaA), R. miehei lipase, A. oryzae alkaline protease, A. oryzae triose phosphate isomerase, A. nidulans acetamidase, the NA2-tpi promoter (a hybrid of the promoters from the genes encoding A. niger neutral alpha-amylase and A. oryzae triose phosphate isomerase), and mutant, truncated, and hybrid promoters thereof. Particularly preferred promoters for use in filamentous fungal cells are a promoter, or a functional part thereof, from a protease gene; e. g., from the F. oxysporum trypsin-like protease gene (U.S. Pat. No. 4,288,627), A. oryzae alkaline protease gene (alp), A. niger pacA gene, A. oryzae alkaline protease gene, A. oryzae neutral metalloprotease gene, A. niger aspergillopepsin protease pepA gene, or F. venenatum trypsin gene, A. niger aspartic protease pepB gene. Other preferred promoters are the promoters described in WO2006/092396 and WO2005/100573, which are herein incorporated by reference.

The term “operably linked” is defined herein as a configuration in which a control sequence is appropriately placed at a position relative to a nucleic acid sequence such that the control sequence facilitates the transcription of a nucleic acid sequence and/or polypeptide.

When the polypeptide according to the invention is a chimeric polypeptide, being comprised of two or more parts of repeat proteins, as described earlier herein, the person skilled in the art knows how to construct these and other chimeric polynucleotide constructs using methods known in the art.

When the polypeptide according to the invention is to be secreted from the host cell into the cultivation medium, an appropriate signal sequence can be added to the polypeptide in order to direct the de novo synthesized polypeptide to the secretion route of the host cell. Appropriate signal sequences are described earlier herein. The person skilled in the art knows how to clone the polynucleotide sequence encoding an appropriate signal sequence in frame with the polynucleotide encoding the polypeptide according to the invention.

As an alternative for a signal sequence, the polypeptide of the invention can be fused to a secreted carrier protein, or part thereof. The skilled person knows how to construct these and other chimeric polynucleotide constructs using methods known in the art. Examples of carrier proteins are described earlier herein.

In order to facilitate expression and/or translation, the polynucleotide or the nucleic acid construct according to the invention may be comprised in an expression vector such that the polynucleotide of the invention is operably linked to the appropriate control sequences for expression and/or translation in vitro, or in prokaryotic or eukaryotic host cells.

The recombinant expression vector may be any vector (e.g., a plasmid or virus), which can be conveniently subjected to recombinant DNA procedures and can bring about the expression of the nucleic acid sequence encoding the polypeptide. The choice of the vector will typically depend on the compatibility of the vector with the host cell into which the vector is to be introduced. The vectors may be linear or closed circular plasmids. The vector may be an autonomously replicating vector, i. e., a vector, which exists as an extra-chromosomal entity, the replication of which is independent of chromosomal replication, e.g., a plasmid, an extra-chromosomal element, a mini-chromosome, or an artificial chromosome. An autonomously maintained cloning vector may comprise the AMA1-sequence (see e.g. Aleksenko and Clutterbuck (1997), Fungal Genet. Biol. 21: 373-397).

Alternatively, the vector may be one which, when introduced into the host cell, is integrated into the genome and replicated together with the chromosome(s) into which it has been integrated. The integrative cloning vector may integrate at random or at a predetermined target locus in the chromosomes of the host cell. In a preferred embodiment of the invention, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearized prior to transformation of the cell. Linearization is preferably performed such that at least one but preferably either end of the cloning vector is flanked by sequences homologous to the target locus. The length of the homologous sequences flanking the target locus is preferably at least 30 bp, preferably at least 50 bp, preferably at least 0.1 kb, even preferably at least 0.2 kb, more preferably at least 0.5 kb, even more preferably at least 1 kb, most preferably at least 2 kb. Preferably, the efficiency of targeted integration into the genome of the host cell, i.e. integration in a predetermined target locus, is increased by augmented homologous recombination abilities of the host cell. Such phenotype of the cell preferably involves a deficient ku70 gene as described in WO2005/095624. WO2005/095624 discloses a preferred method to obtain a filamentous fungal cell comprising increased efficiency of targeted integration. Preferably, the homologous flanking DNA sequences in the cloning vector, which are homologous to the target locus, are derived from a highly expressed locus meaning that they are derived from a gene, which is capable of high expression level in the host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1). A number of preferred highly expressed fungal genes are given by way of example: the amylase, glucoamylase, alcohol dehydrogenase, xylanase, glyceraldehyde-phosphate dehydrogenase or cellobiohydrolase (cbh) genes from Aspergilli or Trichoderma. Most preferred highly expressed genes for these purposes are a glucoamylase gene, preferably an A. niger glucoamylase gene, an A. oryzae TAKA-amylase gene, an A. nidulans gpdA gene, a Trichoderma reesei cbh gene, preferably cbh1.

More than one copy of a nucleic acid sequence may be inserted into the cell to increase production of the gene product. This can be done, preferably by integrating into its genome copies of the DNA sequence, more preferably by targeting the integration of the DNA sequence at one of the highly expressed locus defined in the former paragraph. Alternatively, this can be done by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent. To increase even more the number of copies of the DNA sequence to be over expressed the technique of gene conversion as described in WO98/46772 may be used.

The vector system may be a single vector or plasmid or two or more vectors or plasmids, which together contain the total DNA to be introduced into the genome of the host cell, or a transposon.

The vectors preferably contain one or more selectable markers, which permit easy selection of transformed cells. A selectable marker is a gene the product of which provides for biocide or viral resistance, resistance to heavy metals, prototrophy to auxotrophs, and the like. A selectable marker for use in a filamentous fungal cell may be selected from the group including, but not limited to, amdS (acetamidase), argB (ornithine carbamoyltransferase), bar (phosphinothricinacetyltransferase), bleA (phleomycin binding), hygB (hygromycinphosphotransferase), niaD (nitrate reductase), pyrG (orotidine-5′-phosphate decarboxylase), sC (sulfate adenyltransferase), and trpC (anthranilate synthase), as well as equivalents from other species. Preferred for use in an Aspergillus and Penicillium cell are the amdS (EP 635574 B1, WO 97/06261) and pyrG genes of A. nidulans or A. oryzae and the bar gene of Streptomyces hygroscopicus. More preferably an amdS gene is used, even more preferably an amdS gene from A. nidulans or A. niger. A most preferred selection marker gene is the A.nidulans amdS coding sequence fused to the A.nidulans gpdA promoter (see EP 635574 B1). Other preferred AmdS markers are those described in WO2006/040358. AmdS genes from other filamentous fungi may also be used (WO 97/06261).

The procedures used to ligate the elements described above to construct the recombinant expression vectors of the present invention are well known to one skilled in the art (see, e.g. Sambrook & Russell, Molecular Cloning: A Laboratory Manual, 3rd Ed., CSHL Press, Cold Spring Harbor, N.Y., 2001; and Ausubel et al., Current Protocols in Molecular Biology, Wiley InterScience, NY, 1995).

In a fourth aspect of the present invention, there is provided a host cell comprising the polynucleotide according to the invention or the nucleic acid construct according to the invention.

The host cell according to the invention may be any host cell. For specific uses of the polypeptide according to the invention or the recombinant peptide of interest according to the invention, the selection of host cell may be made according to such use. Where e.g. the polypeptide according to the invention or the recombinant peptide of interest according to the invention is to be used in food applications, a host cell may be selected from a food-grade organism such as Saccharomyces cerevisiae. Specific uses include, but are not limited to, food, (animal) feed, pharmaceutical, agricultural such as crop-protection, and/or personal care applications.

Accordingly, compositions for use in food-, (animal) feed-, pharmaceutical-, agricultural- such as crop-protection, and/or personal care applications, comprising a for the purpose acceptable helper compound and a host cell according to the invention are provided by the present invention.

According to an embodiment, the host cell according to the invention is a eukaryotic host cell. Preferably, the eukaryotic cell is a mammalian, insect, plant, fungal, or algal cell. Preferred mammalian cells include e.g. Chinese hamster ovary (CHO) cells, COS cells, 293 cells, PerC6 cells, and hybridomas. Preferred insect cells include e.g. Sf9 and Sf21 cells and derivatives thereof. More preferably, the eukaryotic cell is a fungal cell, i.e. a yeast cell, such as K. lactis, S. cerevisiae, Hansenula polymorpha, and Pichia pastoris, or a filamentous fungal cell. Most preferably, the eukaryotic cell is a filamentous fungal cell.

“Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, and Trichoderma.

Preferred filamentous fungal cells belong to a species of an Aspergillus, Penicillium or Trichoderma genus, and most preferably a species of Aspergillus niger, Aspergillus sojae, Aspergillus fumigatus, Aspergillus oryzae, Trichoderma reesei or Penicillium chrysogenum.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL) Aspergillus niger CBS 513.88, Aspergillus oryzae ATCC 20423, IFO 4177, ATCC 1011, ATCC 9576, ATCC14488-14491, ATCC 11601, ATCC12892, P. chrysogenum CBS 455.95, Penicillium citrinum ATCC 38065, Penicillium chrysogenum P2, Acremonium chrysogenum ATCC 36225 or ATCC 48272, Trichoderma reesei ATCC 26921 or ATCC 56765 or ATCC 26921, Aspergillus sojae ATCC11906, Chrysosporium lucknowense ATCC44006 and derivatives thereof.

According to another embodiment, the host cell according to the invention is a prokaryotic cell. Preferably, the prokaryotic host cell is bacterial cell. The term “bacterial cell” includes both Gram-negative and Gram-positive microorganisms. Suitable bacteria may be selected from e.g. Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces. Preferably, the bacterial cell is selected from the group consisting of B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Paracoccus denitrificans, E. coli, C. glutamicum, Staphylococcus carnosus, Streptomyces lividans, Sinorhizobium melioti and Rhizobium radiobacter.

In a fifth aspect of the present invention there is provided a process for obtaining a polynucleotide encoding a recombinant polypeptide comprising at least one repeat domain, further comprising a recombinant peptide of interest, comprising:
providing a first polynucleotide encoding a polypeptide comprising at least one repeat domain, said repeat domain comprising at least one repeat unit,
identifying the part(s) of said first polynucleotide encoding said repeat unit, and
inserting into at least one part encoding a repeat unit, a second polynucleotide encoding a recombinant peptide of interest.

According to an embodiment of the invention, the first polynucleotide encodes a polypeptide that is a naturally occurring parental polypeptide according to the first aspect of the invention. Preferably, the naturally occurring polypeptide is a known repeat protein. Preferred repeat proteins are those described earlier herein.

According to another embodiment of the invention, the first polynucleotide encodes a variant of the naturally occurring parental polypeptide.

The first polynucleotide may be provided for by general methods known to the person skilled in the art. Such methods are extensively described in Sambrook & Russell, Molecular Cloning: A Laboratory Manual, 3rd Ed., CSHL Press, Cold Spring Harbor, N.Y., 2001; and Ausubel et al., Current Protocols in Molecular Biology, Wiley InterScience, NY, 1995. Examples of said methods are following. When the sequence of the first polypeptide is already known, or when the sequence of the polypeptide encoded is already known, the polynucleotide may be isolated from a host cell that natively expresses the polynucleotide. Alternatively, the polynucleotide may be synthesized chemically. Codon optimization methods as e.g. described earlier herein may be used for adaptation of the codon use a host cell of choice. If the sequence of the polypeptide is not known, the sequence may first be determined using methods known in the art (Sambrook & Russell; Ausubel, supra).

The identification of the part(s) of the first polynucleotide encoding a repeat unit may be performed using methods known in the art. In most cases this will involve the analysis of the folding topology of the polypeptide encoded. In most cases, the repeat units will exhibit a high degree of sequence identity (same amino acid residues at corresponding positions) or sequence similarity (amino acid residues being different, but having similar physicochemical properties), and some of the amino acid residues might be key residues being strongly conserved in the different repeat units found in naturally occurring proteins.

However, a high degree of sequence variability by amino acid insertions and/or deletions, and/or substitutions between the different repeat units found in naturally occurring proteins will be possible as long as the common folding topology is maintained.

Methods for directly determining the folding topology of repeat proteins by physicochemical means such as X-ray crystallography, NMR or CD spectroscopy, are well known to the practitioner skilled in the relevant art. Methods for identifying and determining repeat units or repeat sequence motifs or for identifying families of related proteins comprising such repeat units or motifs, such as PFAM motifs searches (Finn et al., Pfam: clans, web tools and services. 2006, Nucleic Acids Res. Database Issue 34:D247-D251) and homology searches (BLAST etc.) are well established in the field of bioinformatics, and are well known to the practitioner in such art. The step of refining an initial repeat sequence motif may comprise an iterative process.

The second polynucleotide encoding the recombinant peptide of interest may be provided by methods known in the art (Sambrook & Russell; Ausubel, supra). The polynucleotide may be synthesized chemically or may be isolated or produced by methods known in the art or combinations of such methods; examples of such methods are: PCR, isolation from a host cell, digestion from a parental polynucleotide etc. Codon optimization methods as e.g. described earlier herein may be used for adaptation of the codon use to match most optimally a host cell of choice.

The polynucleotide may be inserted into the first polynucleotide using general cloning techniques as known in the art (Sambrook & Russell; Ausubel, supra). Examples are digestion, ligation, PCR etc.

Alternatively, to inserting the polynucleotide encoding the recombinant peptide of interest, the entire polynucleotide encoding the polypeptide comprising a recombinant polypeptide may be synthesized chemically. Codon optimization methods as e.g. described earlier herein may be used for adaptation of the codon use a host cell of choice.

In order to facilitate introduction into a host cell, the polynucleotide encoding the polypeptide comprising a recombinant polypeptide or the nucleic acid construct comprising said polynucleotide may be comprised in an expression vector such that the polynucleotide of the invention is operably linked to the appropriate control sequences for expression and/or translation. The features of such nucleic acid construct and expression vector are preferably those as described earlier herein.

In a sixth aspect of the present invention there is provided a process for obtaining the host cell comprising the polynucleotide encoding the polypeptide according to the invention, said polypeptide further comprising a recombinant peptide of interest according to the invention, said process comprising:
providing a suitable host cell, and
transforming said host cell with said polynucleotide, nucleic acid construct or expression vector.

The suitable host cell may be a prokaryotic cell, or may be a eukaryotic cell. Preferably, the suitable host cell is the host cell as described earlier herein.

Transformation of the host cell by introduction of a polynucleotide an expression vector or a nucleic acid construct into the cell is preferably performed by techniques well known in the art (see Sambrook & Russell; Ausubel, supra). Transformation may involve a process consisting of protoplast formation, transformation of the protoplasts, and regeneration of the cell wall in a manner known per se. Suitable procedures for transformation of Aspergillus cells are described in EP 238 023 and Yelton et al., 1984, Proceedings of the National Academy of Sciences USA 81:1470-1474. Suitable procedures for transformation of Aspergillus and other filamentous fungal host cells using Agrobacterium tumefaciens are described in e.g. De Groot et al., Agrobacterium tumefaciens-mediated transformation of filamentous fungi. Nat Biotechnol. 1998, 16:839-842. Erratum in: Nat Biotechnol 1998 16:1074. A suitable method of transforming Fusarium species is described by Malardier et al., 1989, Gene 78:147156 or in WO 96/00787. Other methods can be applied such as a method using biolistic transformation as described in: Christiansen et al., Biolistic transformation of the obligate plant pathogenic fungus, Erysiphe graminis f.sp. hordei. 1995, Curr Genet. 29:100-102. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

In a seventh aspect of the present invention there is provided a process for the production of a polypeptide comprising a recombinant peptide according to the invention.

The polypeptide comprising a recombinant peptide according to the invention may be produced by any means known to the person skilled in the art. Such methods include chemical synthesis and in vitro or in vivo expression and translation of the polypeptide or parts thereof. Parts of the polypeptide may be produced by different means known in the art, e.g. part of the polypeptide may be synthesized chemically and part may be produced by expression.

According to the invention, the polypeptide comprising a recombinant peptide according to the invention may be produced by expression of:
the polynucleotide according to the invention, said polynucleotide encoding a polypeptide, comprising at least one repeat domain, said polypeptide further comprising a recombinant peptide of interest,
or the polynucleotide obtained according to the invention by the process of:
(i) providing a first polynucleotide encoding a polypeptide comprising at least one repeat domain, said repeat domain comprising at least one repeat unit,
(ii) identifying the part(s) of said first polynucleotide encoding said repeat unit, and
(iii) inserting into at least one part encoding a repeat unit, a second polynucleotide encoding the recombinant peptide of interest.

According to the invention, the expression of said polynucleotide may be performed in an in vitro expression and translation system system. Such systems are known to the person skilled in the art (see Sambrook & Russell; Ausubel, supra), and may e.g. be a rabbit reticulo lysate based system.

According to the invention, the expression of said polynucleotide may be performed in a host cell transformed with said polynucleotide. The host cell may be any host cell described previously herein.

According to the invention, the polypeptide comprising a recombinant peptide according to the invention may be produced by a process comprising:
culturing the host cell according to the invention, said host cell comprising the polynucleotide according to the invention, said polynucleotide encoding a polypeptide, comprising at least one repeat domain, said polypeptide further comprising a recombinant peptide of interest, under conditions conducive to the expression of the polypeptide, and
optionally purifying the polypeptide.

According to the invention, the host cells may be cultivated in a nutrient medium suitable for production of the polypeptide according to the invention using methods known in the art. For example, the cells may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art (for filamentous fungal hosts see, e. g., Bennett, J. W. and LaSure, L., eds., More Gene Manipulations in Fungi, Academic Press, CA, 1991). Suitable media are available from commercial suppliers or may be prepared using published compositions (e. g., in catalogues of the American Type Culture Collection). If the polypeptide is secreted into the nutrient medium, the polypeptide can be recovered directly from the medium. If the polypeptide is not secreted, it is recovered from cell lysates.

The resulting polypeptide may be isolated by methods known in the art. For example, the polypeptide may be isolated from the nutrient medium by conventional procedures including, but not limited to, centrifugation, filtration, extraction, spray drying, evaporation, or precipitation. The isolated polypeptide may then be further purified by a variety of procedures known in the art including, but not limited to, chromatography (e. g., ion exchange, affinity, hydrophobic, chromatofocusing, and size exclusion), electrophoretic procedures (e.g., preparative isoelectric focusing, differential solubility (e. g., ammonium sulfate precipitation), or extraction (see, e.g., Protein Purification, J.-C. Janson and Lars Ryden, editors, VCH Publishers, New York, 1989). Specific uses may require specific purification methods. The skilled person knows how to select and perform the purification methods known in the art, e.g. it is generally known that a pharmaceutical application of a peptide and/or polypeptide requires pharmaceutical grade compounds and thus purification to pharmaceutical grade.

The recombinant peptide according to the invention may conveniently be used when still present in the polypeptide according to the invention if the trait of interest, preferably a biological activity, is present in this form. In this case, the process of production of the recombinant peptide is analogous to the process for the production of the polypeptide according to the invention.

Alternatively, the recombinant peptide may be isolated from the polypeptide. The isolation may result in isolation of the recombinant peptide of interest per se. The isolation may also result in the isolation of a peptide fragment comprising the recombinant peptide of interest. The isolation may also result in the isolation of the recombinant peptide and/or a peptide fragment comprising the recombinant peptide of interest.

According to the invention, the recombinant peptide according to the invention may thus be produced by a process comprising:
culturing the host cell according to the invention, said host cell comprising the polynucleotide according to the invention, said polynucleotide encoding a polypeptide, comprising at least one repeat domain, said polypeptide further comprising a recombinant peptide of interest, under conditions conducive to the expression of the polypeptide,
optionally purifying the polypeptide, and
isolating the recombinant peptide and/or a peptide fragment comprising the recombinant peptide from the polypeptide.

Specific uses of the recombinant peptide according to the invention and/or peptide fragment comprising the recombinant peptide of interest include, but are not limited to, food, (animal) feed, pharmaceutical, agricultural such as crop-protection, and/or personal care applications. Examples of peptides used in these applications are those described earlier herein.

Accordingly, compositions for use in food-, (animal) feed-, pharmaceutical-, agricultural- such as crop-protection, and/or personal care applications, comprising a for the purpose acceptable helper compound and a recombinant peptide according to the invention or a peptide fragment comprising the recombinant peptide according to the invention are provided by the present invention. Furthermore, the recombinant peptide according to the invention and/or a peptide fragment comprising the recombinant peptide according to the invention for use as a medicament is provided by the invention.

Release of the recombinant peptide or a peptide fragment comprising the recombinant peptide of interest may be performed by methods known in the art. For instance, the recombinant peptide(s) may be released by digestion of the polypeptide of the invention with an appropriate proteolytic enzyme. Such method is known from the prior art for the production of peptides involving the hydrolysis of polypeptides or polypeptide mixtures wherein a peptide of interest is present, whereby the peptide of interest is released from the polypeptide. For example, the anti-hypertensive peptide XPP, wherein “X” may be any natural amino acid, is released from casein after processing with an Aspergillus oryzae protease (Mizuno et al., Release of Short and Proline-Rich Antihypertensive Peptides from Casein Hydrolysate with an Aspergillus oryzae Protease. 2004, J. Dairy Sci. 87:3183-3188). As such, the method described by Mizuno et al. for the production of a peptide has the disadvantage that the peptide is contaminated with a large amount of other peptides and polypeptide fragments, due to the unfavourable ratio between peptide of interest versus polypeptide. The present invention however, conveniently allows shifting the ratio to a more favourable one by comprising a large copy number of recombinant peptides into the polypeptide according to the invention.

If the trait of interest of the recombinant peptide is sufficiently present, no purification is necessary. If isolation is required, it may be performed by any process known to the skilled person.

When the recombinant peptide is flanked on the C-terminal or the N-terminal side by an amino acid motif facilitating isolation of the recombinant peptide, the peptide may be released by a specific releasing agent. The releasing agent may be a proteolytic enzyme or chemical agent. Examples of proteolytic enzymes are described in http://www.expasy.ch/tools/peptidecutter/. An example in which a peptide flanked by PX (X=any amino acid) motifs is isolated after proteolytic cleavage with proline specific protease is described in WO2006/089921 and a peptide flanked by KX motifs that is isolated after trypsin digestions is described in Prak et al., Design of genetically modified soybean proglycinin A1aB1b with multiple copies of bioactive peptide sequences. 2006, Peptides. 27:1179-1186. An example of chemical processing of a polypeptide containing peptides flanked by MX motifs for cleavage by Cyanogen Bromide is described in Metlitskaia et al., Recombinant antimicrobial peptides efficiently produced using novel cloning and purification processes. 2004, Biotechnol. Appl. Biochem. 39:339-345. Alternatively, the recombinant peptide is released from the polypeptide by autoprocessing. Autoprocessing is defined as cleaving activity which a polypeptide carries out itself. For example, the polypeptide is cleaved by a protease located in the polypeptide or the polypeptide contains sequences that mediate cleavage within the ribosome during translation, such as the foot-and-mouth disease virus 2A region (De Felipe et al., Co-translational, intraribosomal cleavage of polypeptides by the foot-and-mouth disease virus 2A peptide, 2003, J. Biol. Chem. 278:11441-11448).

In an eight aspect of the present invention there is provided the use of a repeat protein for the production of a recombinant peptide of interest. Preferably, the recombinant peptide is the peptide described earlier herein.

According to the invention, the repeat protein comprises at least one recombinant peptide of interest. Preferably, said peptide is comprised in a repeat domain. More preferably, said peptide is comprised in a repeat unit. The repeat protein may comprise one or more repeat domains and/or may comprise one or more repeat units. The recombinant peptide may be present in one or more repeat units. The recombinant peptide may be present in one or more repeat domains. The recombinant peptide may be present in all repeat units. The recombinant peptide may be present in dimers, trimers and other multimers. According to the invention, distinct recombinant peptides may be present in a repeat unit.

The repeat protein may be any repeat protein. Repeat proteins are known to the skilled person and these may conveniently applied for use in the invention. Repeat proteins are reviewed by Main E R et al. (Main et al, The folding and design of repeat proteins: reaching a consensus. 2003, Curr Opin Struct Biol. 13:482-489); the design of repeat proteins is inter alia described in Binz et al., Designing repeat proteins: well-expressed, soluble and stable proteins from combinatorial libraries of consensus ankyrin repeat proteins. 2003, J Mol Biol. 332:489-503; Kohl et al., Designed to be stable: crystal structure of a consensus ankyrin repeat protein. 2003, Proc Natl Acad Sci USA. 100:1700-1705. In WO02/20565, recombinant modulation of repeat proteins is described to construct recombinant binding proteins. No production of a peptide of interest is described. However, the general knowledge on repeat proteins and the techniques used for the modulation of the repeat proteins of WO02/20565 can conveniently be used in the invention and WO02/20565 is herein incorporated by reference.

The repeat protein comprising a recombinant peptide may be a polypeptide according to the invention as described herein. Preferably, the polypeptide according to the invention is derived from a naturally occurring parental polypeptide. More preferably, the naturally occurring polypeptide is a known repeat protein. Preferred naturally occurring repeat proteins are those as described earlier herein.

The production of the recombinant peptide may be performed by the methods according to the invention as described herein, e.g. a repeat protein comprising a recombinant polypeptide may be produced by culturing a host that produces the repeat protein, followed by optional purification of the protein and optional release and purification of the recombinant polypeptide.

The invention described and claimed herein is not to be limited in scope by the specific embodiments herein enclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In case of conflict, the present disclosure including definitions will be taken as a guide.

### EXAMPLES

### General Materials and Methods

In the examples described herein, standard molecular cloning techniques such as isolation and purification of nucleic acids, electrophoresis of nucleic acids, host cell transformations, enzymatic modification, cleavage and/or (quantitative) amplification of nucleic acids, Western blotting etc., were performed as described in the handbooks well known to the person skilled in the art (see, e.g., Sambrook & Russell, “Molecular Cloning: A Laboratory Manual”, 3rd Ed., CSHL Press, Cold Spring Harbor, N.Y., 2001; and Ausubel et al., “Current Protocols in Molecular Biology”, Wiley InterScience, NY, 1995; and Innis et al. (eds.) (1990) “PCR protocols, a guide to methods and applications” Academic Press, San Diego).

Identification of Repeat Proteins with Variable Loops

Using Genedata software (Basel, Switzerland), the Aspergillus niger genome of CBS 513.88 (Pel et al., Genome sequencing and analysis of the versatile cell factory Aspergillus niger CBS 513.88. 2007, Nat Biotechnol. 25:221-31) was screened for proteins containing diverse repeat protein PFAM motifs (Finn et al., Pfam: clans, web tools and services. 2006, Nucleic Acids Res. Database Issue 34:D247-D251). The existence of variable loops in repeat units was analyzed manually.

### Protein Analysis

Protein samples were separated under reducing conditions on NuPAGE 4-12% Bis-Tris gel (Invitrogen, Breda, The Netherlands). For SYPRO Ruby staining, gels were fixed by incubating 2 times 30 minutes in 10% methanol+7% acetic acid and stained overnight with SYPRO Ruby protein gel stain (Molecular Probes, Breda, The Netherlands). After washing in 10% methanol+7% acetic acid, protein bands were visualized using a Typhoon scanner (GE Healthcare, Den Bosch, the Netherlands).

For Western blotting, proteins were transferred to nitrocellulose. The nitrocellulose filter was blocked with TBST (Tris buffered saline containing 0.1% Tween 40) containing 3% skim-milk and incubated for at least 2 hours with anti-polyHis antibody (Sigma, Zwijndrecht, The Netherlands) or anti-FLAG M2 antibody (Sigma, Zwijndrecht, The Netherlands). Blots were washed twice with TBST for 10 minutes and stained with Horse-radish-peroxidase conjugated rabbit-anti-mouse antibody (DAKO, Glostrup, Denmark) for 1 hours. After washing the blots five times with TBST for 10 minutes, proteins were visualized using SuperSignal (Pierce, Rockford, U.S.A).

### Example 1

### Expression of Ankyrin Repeat Protein Containing Recombinant Peptide IPP in E. Coli

This example describes the cloning and expression of the E3_{—}5 ankyrin repeat protein according to SEQ ID NO: 1 and 2 and of the E3_{—}5 ankyrin repeat protein containing a recombinant IPP peptide according to SEQ ID NO: 3 and 4.

### Defining Variable Loop Within Ankyrin Repeat Units

The variable loop within ankyrin repeat units of the E3_{—}5 ankyrin repeat protein was determined as described in “General Materials and Methods”. Within ankyrin repeat units, the variable loop is located in between the second alpha-helix of unit 1 and the first alpha-helix of the C-terminal adjacent unit.

Cloning of E.coli Expression Plasmids pGBE01Ank1 Encoding Ankyrin Repeat Protein and pGBE01Ank2 Encoding Ankyrin Repeat Proteins Containing IPP

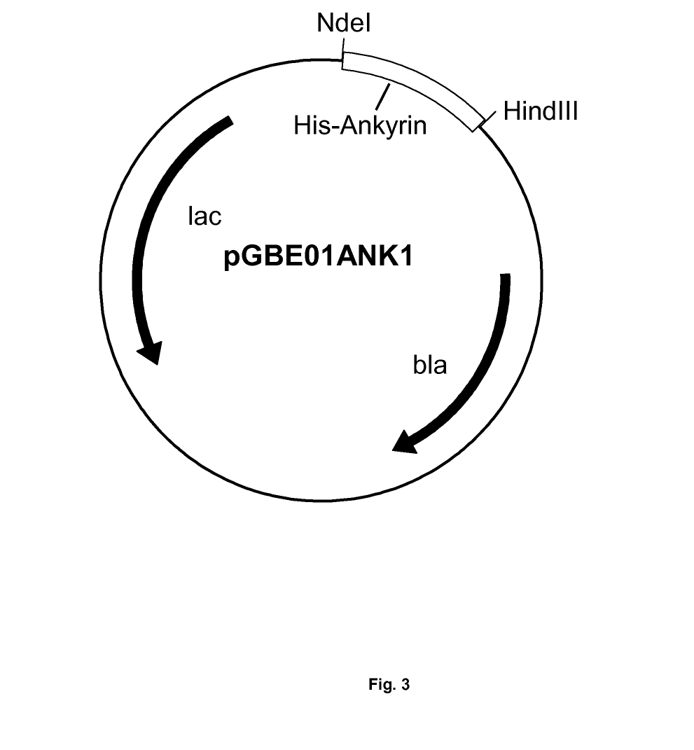

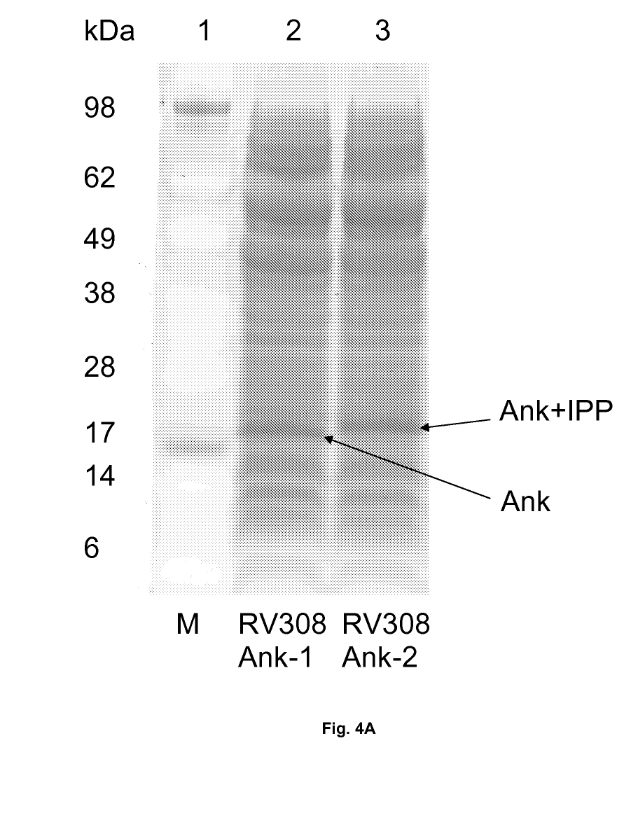

Synthetic ankyrin repeat protein E3_{—}5 (GenBank accession number AY195853, Kohl et al, 2003, Designed to be stable: crystal structure of a consensus ankyrin repeat protein. Proc Natl Acad Sci USA. 100:1700-1705), a synthetic ankyrin repeat protein consisting of 5 ankyrin repeat units and a C-terminal His-tag was used as model ankyrin repeat protein. The E3_{—}5 gene and a variant containing recombinant peptide IPP were synthesised at Sloning Biotechnology (Puchheim, Germany) and cloned as NdeI/HindIII fragment into E.coli expression plasmid pMS470 containing the tac promoter (Balzer D, Ziegelin G, Pansegrau W, Kruft V, Lanka E. KorB protein of promiscuous plasmid RP4 recognizes inverted sequence repetitions in regions essential for conjugative plasmid transfer. 1992, Nucleic Acids Res. 20, 1851-1858). FIG. 3 represents a map of pGBE01Ank1 containing the E3_{—}5 coding region (E3_{—}5 coding region represented by SEQ ID NO: 2) under control of the tac promoter within vector pMS470. Figure is representative for pGBE01Ank2, which plasmid comprises the E3_{—}5 coding region+recombinant IPP (SEQ ID NO: 4). Recombinant IPP was introduced in the variable loop of ankyrin repeat unit 3 (amino acid 109-111 in SEQ ID NO: 3). A list of E.coli expression plasmids, the constructs comprised in the plasmids and the SEQ ID NO's of the constructs are depicted in Table 1.

TABLE 1
E. coli expression plasmids:

| | | Sequence | |
|---|---|---|---|
| | | identity | Sequence |
| | | of nucleic acid | identity of |
| Plasmid | Construct in plasmid | construct | protein |
| pGBE01Ank1 | E3_5 ankyrin repeat | SEQ ID NO: 2 | SEQ ID NO: 1 |
| | protein | | |
| pGBE01Ank2 | E3_5 ankyrin repeat | SEQ ID NO: 4 | SEQ ID NO: 3 |
| | protein + IPP | | |

Preparation of E. coli Lysates

Typically, 2 ml of E. coli cultures expressing ankyrin repeat protein with and without recombinant peptide IPP were centrifuged and cell pellets were lysed in 400 μl B-PER II Bacterial protein extraction buffer (Pierce, Rockford, U.S.A) containing EDTA-free protease inhibitor cocktail (Roche, Almere, The Netherlands). The suspension was incubated for 60 minutes in rotary mixer at room temperature and centrifuged at 16.100×g for 15 minutes. Supernatants were collected and analyzed by SDS-PAGE.

Transformation of E. coli

E.coli strain RV308 (ATCC31608) was transformed with either pGBE01Ank1 or pGBE01Ank2. The procedures used to transform E.coli with expression plasmids were according to “General Materials and Methods”. A list of E.coli strains and the plasmid present in the transformant is depicted in Table 2. Representative transformants were selected for further analysis.

TABLE 2
E. coli strains:

| strain | plasmid |
|---|---|
| RV308-Ank1 | pGBE01Ank1 (Ankyrin) |
| RV308-Ank2 | pGBE01Ank2 (Ankyrin + IPP) |

Expression of pGBE01Ank1 and pGBE01Ank2 in Escherichia coli

E.coli strains RV308-Ank1 and RV308-Ank2 were inoculated in trypton yeast (2*TY) broth containing 100 μg/ml ampicillin and incubated at 28° C. under continuous shaking for 16 hours. Cultures were diluted to OD 0.015 at 610 nm in 2*TY medium containing 100 μg/ml ampicillin and incubated at 28° C. under continuous shaking. When cultures reached the optimal density of OD 1.0 at 610 nm, the tac promoter was induced using 0.5 mM isopropyl-beta-D-thiogalactopyranoside (IPTG, Fluka, Buchs, Switzerland). Cells were harvested after 16 hours incubation at 28° C. under continuous shaking.

### Expression Analysis

Lysates of RV308-Ank1 and RV308-Ank2 were separated on SDS-PAGE and visualized either by SYPRO Ruby protein gel stain or Western blotting as described in “General Materials and Methods”.

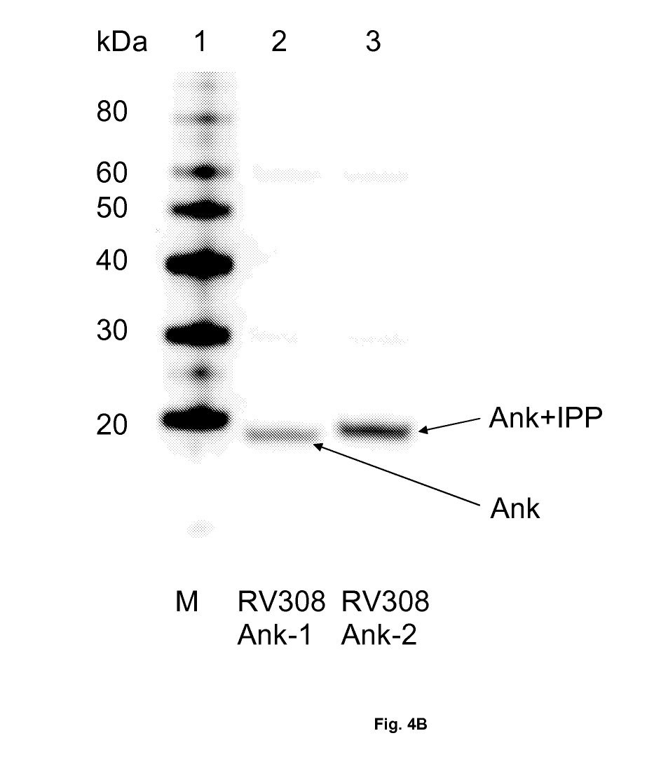

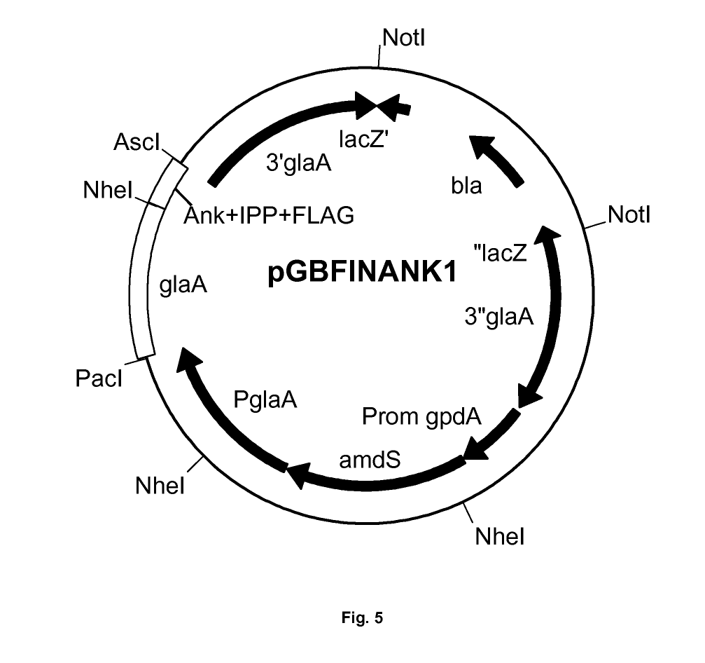

The results of the SYPRO Ruby protein gel stain and Western blotting are presented in FIGS. 4A and 4B, respectively. Both on the SYPRO Ruby stained gel and Western blot specific protein bands of about 18 kDa were observed, the expected molecular weight for Ankyrin repeat protein. Approximately, equal amounts of ankyrin repeat protein was present in lysates of RV308-Ank1 (control) and RV308-Ank2 (Ank+IPP), indicating that ankyrin repeat protein comprising recombinant peptide IPP was successfully expressed and that insertion of IPP with ankyrin repeat protein did not affect expression and stability of the protein. Thus, a recombinant peptide was stably inserted in an ankyrin repeat protein and successfully expressed.

### Example 2

Expression of a Chimeric Glucoamylase-Ankyrin Repeat Protein Containing IPP in A. niger

This example describes the expression of a chimeric glucoamylase-ankyrin repeat protein, containing IPP in the ankyrin fragment; the ankyrin fragment being fused to glucoamylase to enable secretion of the chimeric protein.

### Cloning of Expression Plasmid Encoding Glucoamylase-Ankyrin Repeat Protein+IPP.

For expression studies in A.niger, the encoding region of the ankyrin repeat protein comprising IPP was obtained by PCR using pGBE01Ank2 as template. Restriction sites and the DNA sequence encoding the C-terminal FLAG-tag were included in the primers. Primers were synthesized by Invitrogen (Breda, The Netherlands). The following PCR primers were used:

NheI_EcAnk-For (SEQ ID NO: 9):
CTAGCTAGCAAGTCCGACCTGGGTAAGAAACTGC;
and
FLAG_Asc_EcAnk-Rev (SEQ ID NO: 10):
GGCGCGCCTTTACTTGTCATCATCATCCTTGTAGTCTTGCAGGATTTCAG
CCAGGTCC

NheI and AscI restriction sites for cloning purposes are depicted in italics, sequences homologous to the pGBE01Ank2 template are underlined. The sequence encoding the FLAG-tag is in bold.

PCR conditions for all reactions: 50 μl reaction mix with 2 ng of template DNA, 0.2 μM of each primer, 0.2 mM of dNTPs, 1× Phusion HF buffer and 1U of Phusion DNA-Polymerase, according to Phusion High-Fidelity DNA Polymerase Manual (Finnzymes, Espoo, Finland), 30 s denaturation at 98° C., amplification in 25 cycles (10 s 98° C., 30 s 66° C., 30 s 72° C.), and a final incubation of 10 min at 72° C. The resulting PCR fragment (shown in SEQ ID NO: 5) was purified using the NucleoSpin ExtractII kit (Macherey-Nagel, Easton, USA), and was digested with NheI and AscI.

The glucoamylase encoding part of the chimeric glucoamylase-ankyrin repeat fusion construct, including PacI and NheI restriction sites, Kozak sequence and ATG, was synthesized by Sloning Biotechnology (Puchheim, Germany) (shown in SEQ ID NO: 6).

The glucoamylase fragment was purified using the NucleoSpin ExtractII kit (Macherey-Nagel, Easton, USA).

pGBFINAnk1, which is depicted in FIG. 5, was constructed by inserting the PacI/NheI glucoamylase fragment and the NheI/AscI chimeric ankyrin repeat PCR fragment via a 3-point ligation into pGBFIN-5, which plasmid is described in WO 9932617. The pGBFIN5 expression vector comprises the glucoamylase promoter, cloning site, terminator region, an amdS marker operably linked to the gpd promoter, and 3′ and 3″ glaA flanks for targeting. The amino acid and nucleotide sequences of the chimeric glucoamylase-ankyrin repeat fusion is represented by SEQ ID NO: 7 and SEQ ID NO: 8, respectively.

Transformation of Aspergillus niger with pGBFINAnk1

Aspergillus niger WT-1 strain was used for transformations. The Aspergillus niger WT-1 strain is derived from the A.niger strain deposited at the CBS Institute under the deposit number CBS 513.88. It comprises a deletion of the gene encoding glucoamylase (glaA), which was constructed by using the “MARKER-GENE FREE” approach as described in EP 0 635 574. In this patent it is extensively described how to delete glaA specific DNA sequences in the genome of CBS 513.88. The procedure resulted in a MARKER-GENE FREE ΔglaA recombinant A. niger CBS513.88 strain, possessing finally no foreign DNA sequences at all.

In order to introduce the pGBFINAnk1 vector in Aspergillus niger WT-1, transformation and subsequent transformant selection was carried out essentially as described in WO98/46772 and WO99/32617. In brief, linear DNA of the vector was isolated after digestion with NotI, to remove the E.coli sequences from the vector. After transformation, transformants were selected on media comprising acetamide as sole nitrogen source and colony purified. Copy numbers were estimated by quantitative-PCR and low and high copy number transformants were selected. Quantitative PCR was performed as in the “General Materials and Methods”. Single copy and high copy transformants were cultured in shake flasks in 100 ml of CSM-MES medium as described in EP 635 574 at 34° C. at 170 rpm in an incubator shaker using a 500 ml baffled shake flask. After 2 and 3 days of fermentation, supernatant samples were harvested to determine expression by Western blotting using a FLAG-specific antibody. In day 2 and day 3 supernatants of a single copy strain, FLAG-tagged chimeric glucoamylase-ankyrin repeat fusion protein comprising recombinant peptide IPP was detected, indicating that pGBFINAnk1 is expressed in Aspergillus niger.

This experiment clearly demonstrated that a chimeric repeat protein comprising recombinant tripeptide IPP was successfully expressed and secreted using Aspergillus niger as a host cell.

### Example 3

Isolation of Tripeptide IPP and an IPP Containing Peptide From E.coli Transformed with pGBE01ANK2

This example describes the isolation of recombinant tripeptide IPP peptide and an IPP containing peptide out of Ankyrin repeat protein comprising recombinant IPP (Ank2) expressed by E.coli strain RV308-Ank2 (see EXAMPLE 1). Expressed protein was purified, and processed. The presence of IPP in the hydrolysate was determined by mass spectrometry.

Isolation and Processing of IPP Containing His-Tagged-Ankyrine Expressed in E.coli

E.coli transformants RV308-Ank1 and RV308-Ank2 (see EXAMPLE1) were used to inoculate 100 ml of 2*TY broth containing 100 μg/ml ampicillin and protein was expressed as in EXAMPLE 1. The cultures were centrifuged and cell pellets were extracted with 20 ml of B-PER II Bacterial Protein Extraction Reagent (Pierce Prod # 78248, Rockford, U.S.A) for at least 30 minutes under mild stirring. Proteolysis was prevented by adding 0.5 ml of protease inhibitor cocktail for use in purification of Histidine-tagged proteins (Sigma Prod #P8849, Zwijndrecht, The Netherlands).

20 ml of extract was loaded on a HisPur Cobalt Spin Column (Pierce Prod #89969, Rockford, U.S.A) and washed and eluted according to the manufacturer's instructions. Column steps were monitored by SDS-PAGE as described in “General Material and Methods”. Fractions containing purified ankyrin repeat protein were pooled and kept frozen until further use.

To liberate tripeptide IPP, 0.3 ml of purified Ank2 protein (˜0.12 mg) was diluted 5 times with 1.2 ml of 0.1 M acetate buffer pH 4.0 and incubated at 55° C. with 6 mU of a prolyl endoprotease (DSM, WO2006/089921), as described in Edens et al., Extracellular Prolyl Endoprotease from Aspergillus niger and Its Use in the Debittering of Protein Hydrolysates 2005, J. Agric. Food Chem. 53:7950-7957). Liberation of tripeptide IPP was measured by means of LC/MS/MS, using an ion trap (LCQ) mass spectrometer (ThermoFisher®, Breda, the Netherlands) coupled to a P4000 pump (ThermoFisher®, Breda, the Netherlands). The tripeptide IPP was separated from other peptides using a Varian Inertsil 3 u ODS-3, 150*2.1 mm column in combination with a gradient of 0.1% formic acid in LC/MS grade water (Solution A) and 0.1% formic acid in LC/MS grade acetonitrile (Solution B) for elution.

The gradient started at 100% of Solution A, kept here for 5 minutes and increased to 5% of solution B in 15 minutes and was kept at the latter ratio for another 6 minutes. The injection volume used was 50 microliters, the flow rate was 200 microliter per minute and the column temperature was maintained at 55° C. The tripeptide IPP, C₁₆H₂₇N₃O₄, is characterized by a protonated molecule at m/z 326.2 in MS mode, and identified in MS/MS mode according to the peptide fragmentation rules using B- and Y-ion series (nomenclature of Roepstorff and Biemann). Liberation of tripeptide IPP was completed within 1 hour of incubation.

This experiment clearly demonstrated that from the ankyrin repeat protein expressed in E.coli, the recombinant tripeptide IPP was isolated.

To isolate a peptide containing IPP, purified Ank2 protein was diluted 5 times with 0.1 M acetate buffer pH 4.0. Purified Ank1, expressed by strain RV308-Ank1, was used as control. The following solutions were added to 100 microliter of sample (˜0.04 mg of ankyrin repeat protein): 450 microliter of 100 mM NH₄HCO₃, and 20 microliter of trypsin solution (250 microgram/ml). Samples were incubated for 3 hours at 37° C. and the digestion was stopped by adding 6 microliter of PMSF solution (100 mM). After reducing the samples by adding 6 microliter of 100 mM DTT solution, samples were incubated for half an hour at room temperature and analysed by LC/MS/MS, using a linear ion trap-orbitrap (LTQ/orbitrap) mass spectrometer (ThermoFisher®, Breda, the Netherlands) coupled to an Accela pump (ThermoFisher®, Breda, the Netherlands). The IPP containing peptide was separated from other peptides using an Agilent SB C18, 1.8 um 50*2.1 mm column in combination with a gradient of 0.1% formic acid in LC/MS grade water (Solution A) and 0.1% formic acid in LC/MS grade acetonitrile (Solution B) for elution. The gradient started at 95% of Solution A, immediately increasing to 40% of solution B in 13 minutes. The injection volume used was 10 microliter, the flow rate was 400 microliter per minute and the column temperature was maintained at 55° C. After tryptic digestion Ank2, containing IPP, should contain the peptide HGADVNAIPPYDNDGHTPLHLAAK, M=2523.7, which is characterized by multiple charged ions (m/z 1262.5, 842.3, 632.1, 506.0, and 421.8) in MS mode.

For identification MS/MS of the [M+4H]⁴⁺ and [M+5H]⁵⁺ was performed, and the peptide fragmentation rules using B- and Y-ion series (nomenclature of Roepstorff and Biemann), are used for unambiguously identification of the correct amino acid sequence.

Using the above described strategy the peptide HGADVNAIPPYDNDGHTPLHLAAK was identified in trypsin digested Ank2 (Ank+IPP) sample, and was not identified in Ank1 (control without IPP).

This experiment clearly demonstrated that from the ankyrin repeat protein expressed in E.coli, a peptide fragment comprising the recombinant tripeptide IPP was isolated.

### Example 4

Expression of a Designed Chimeric Glucoamylase-Ankyrin Repeat Proteins Containing IPP in A. niger

This example describes the expression of a designed chimeric glucoamylase-ankyrin repeat protein, containing IPP in the ankyrin fragment; the ankyrin fragment being fused to glucoamylase to enable secretion of the chimeric protein. In addition, the example describes the expression of a designed chimeric glucoamylase-KexB-His-ankyrin repeat protein, containing a KexB site in between the glucoamylase fragment and the ankyrin fragment to enable the separate secretion of the glucoamylase and ankyrin.

Cloning of A. niger Expression Plasmids pGBFINAnk11 Encoding a Designed Chimeric Glucoamylase-Ankyrin Repeat Protein Containing IPP and pGBFINAnk12 Encoding a Designed Chimeric Glucoamylase-KexB-His-Ankyrin Repeat Protein Containing IPP

The designed chimeric ankyrin repeat protein Ank11 consists of a glucoamylase fragment fused to an ankyrin repeat fragment of 5 ankyrin repeat units, containing one IPP, and a C-terminal FLAG-tag. The designed chimeric ankyrin repeat protein Ank12, consists of a glucoamylase fragment fused to an ankyrin repeat fragment consisting of 5 ankyrin repeat units, an N-terminal His-tag and containing one IPP, and a C-terminal FLAG-tag. In addition, Ank12 contains a KexB site in between the glucoamylase fragment and the ankyrin fragment. The genes encoding Ank11 and Ank12 were synthesised at Sloning Biotechnology (Puchheim, Germany) and cloned as PacI/AscI fragment into A.niger expression plasmid pGBFIN-5 (described in EXAMPLE2). FIG. 3 is representative for pGBFINAnk11 and pGBFINAnk12. The amino acid and nucleotide sequences of Ank11 are represented by SEQ ID NO: 11 and SEQ ID NO: 12, respectively. The amino acid and nucleotide sequences of Ank12 are represented by SEQ ID NO: 13 and SEQ ID NO: 14, respectively. A list of A.niger expression plasmids, the constructs comprised in the plasmids and the SEQ ID NO's of the constructs are depicted in Table 2.

TABLE 2
A. niger expression plasmids:

| | | Sequence | |
|---|---|---|---|
| | | identity | Sequence |
| | | of nucleic acid | identity of |
| Plasmid | Construct in plasmid | construct | protein |
| pGBFINAnk11 | Designed chimeric ankyrin | SEQ ID NO: 12 | SEQ ID NO: 11 |
| | repeat protein, consisting of | | |
| | glucoamylase, ankyrin | | |
| | fragment + IPP, and FLAG- | | |
| | tag | | |
| pGBFINAnk12 | Designed chimeric ankyrin | SEQ ID NO: 14 | SEQ ID NO: 13 |
| | repeat protein, consisting of | | |
| | glucoamylase fragment, | | |
| | KexB site, His-tag, ankyrin | | |
| | fragment + IPP, and FLAG- | | |
| | tag | | |

Transformation of Aspergillus niger with pGBFINAnk11 and pGBFINAnk12

The transformation of Aspergillus niger was performed as described in EXAMPLE 2. Transformants were grown in shake flasks as described in EXAMPLE 2 and supernatants were harvested at day 2, 3, 4, 5, 6, and 7 to determine expression by Western blotting using a FLAG-specific antibody. In all samples of pGBFINAnk11 transformants, FLAG-tagged chimeric glucoamylase-ankyrin repeat fusion protein comprising recombinant peptide IPP was detected, indicating that pGBFINAnk11 is expressed in Aspergillus niger. In day 2 supernatants of pGBFINAnk12 transformants a clear FLAG-tagged ankyrin protein band was observed without glucoamylase fragment, indicating that pGBFINAnk12 is expressed and that Kex processed ankyrin repeat protein is produced in Aspergillus niger.

This experiment clearly demonstrated that a designed chimeric repeat protein comprising recombinant tripeptide IPP was successfully expressed and secreted using Aspergillus niger as a host cell. In addition, the experiment showed that ankyrin repeat proteins without glucoamylase fragment are produced in Aspergillus niger after Kex processing of the designed chimeric glucoamylase-Kex-His-ankyrin-FLAG protein.

### Example 5

Isolation of Tripeptide IPP from A.niger Transformed with pGBFINANK11

This example describes the isolation of recombinant tripeptide IPP peptide from chimeric glucoamylase-ankyrin repeat protein comprising recombinant IPP (Ank11) expressed by A.niger in EXAMPLE 4. Expressed protein was processed and the presence of IPP in the hydrolysate was determined by mass spectrometry.

Processing of IPP Containing Chimeric Glucoamylase-Ankyrin Repeat Protein Expressed in A.niger

A.niger strains transformed with pGBFINAnk11 (see EXAMPLE 4) were cultured in 100 ml of CSM/MES medium according to the procedure as described in EXAMPLE 2. Supernatant was harvested at days 2, 3, 4, 5 and 6 and was used for processing with prolyl endoprotease according to the procedure as described in EXAMPLE 3. Liberation of tripeptide IPP was measured by means of LC/MS/MS according to the procedure as described in EXAMPLE 3. Free tripeptide IPP, C₁₆H₂₇N₃O₄ was identified in this analysis. Moreover, the amount of free IPP detected correlated with the amount of total protein expressed by A.niger.

This experiment clearly demonstrated that from the chimeric glucoamylase-ankyrin repeat protein expressed by A.niger, the recombinant tripeptide IPP can conveniently be isolated.

1. A polypeptide, comprising at least one repeat domain, further comprising a recombinant peptide of interest.

2. A polynucleotide encoding the polypeptide according to claim 1.

3. A nucleic acid construct comprising the polynucleotide according to claim 2.

4. A host cell comprising the polynucleotide according to claim 2.

5. A process for obtaining the polynucleotide according to claim 2, comprising:
providing a first polynucleotide encoding a polypeptide comprising at least one repeat domain, said repeat domain comprising at least one repeat unit,
identifying the part(s) of said first polynucleotide encoding said repeat unit, and
inserting into at least one part encoding a repeat unit, a second polynucleotide encoding a recombinant peptide of interest.

6. A process for obtaining the host cell according to claim 4, comprising:
providing a suitable host cell, and
transforming said host cell with the polynucleotide.

7. A process for the production of a polypeptide according to claim 1, comprising expression of the polynucleotide according to claim 2, said polynucleotide encoding a polypeptide, comprising at least one repeat domain, said polypeptide further comprising a recombinant peptide of interest.

8. A process for the production of the polypeptide according to claim 1, comprising:
culturing the host cell according to claim 4, said host cell comprising the polynucleotide encoding a polypeptide, comprising at least one repeat domain, said polypeptide further comprising a recombinant peptide of interest, under conditions conducive to the expression of said polypeptide, and
optionally purifying said polypeptide.

9. A process for the production of a recombinant peptide of interest, comprising:
culturing the host cell according to claim 4, said host cell comprising the polynucleotide encoding a polypeptide, comprising at least one repeat domain, said polypeptide further comprising a recombinant peptide of interest, under conditions conducive to the expression of said polypeptide,
optionally purifying said polypeptide, and
isolating said peptide and/or a peptide fragment comprising the recombinant peptide from said polypeptide.

10. A composition for use in food-, (animal) feed-, pharmaceutical-, agricultural- such as crop-protection, and/or personal care applications, comprising:
a for the purpose acceptable helper compound, and
a recombinant peptide obtained by
the polypeptide according to claim 1.

11. The polypeptide according to claim 1, or the polypeptide obtained by the peptide and/or a peptide fragment comprising the recombinant peptide for use as a medicament.

12. Use of a repeat protein for the production of a recombinant peptide.


SEQUENCE LISTING (14 sequences)

SEQ ID NO: 1 - protein, 166 aa, Artificial
  His-tagged Ankyrin repeat protein
MRGSHHHHHHGSDLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQ

SEQ ID NO: 2 - DNA, 501 bp, Artificial
  His-tagged Ankyrin repeat protein
atgagaggatcgcatcaccatcaccatcacggatccgacctgggtaagaaactgctggaagctgctcgtgctggtcaggacgacgaagttcgtatcctgatggctaacggtgctgacgttaacgctactgacaatgatggttatactccgctgcacctggctgcttctaatggtcacctggaaatcgttgaagttctgctgaagaacggtgctgacgttaacgcttctgaccttactggtattactccgctgcacctggctgctgctactggtcacctggaaatcgttgaagttctgctgaagcacggtgctgacgttaacgcttatgacaatgatggtcatactccgctgcacctggctgctaagtatggtcacctggaaatcgttgaagttctgctgaagcacggtgctgacgttaacgctcaggacaaattcggtaagaccgctttcgacatctccatcgacaacggtaacgaggacctggctgaaatcctgcaataa

SEQ ID NO: 3 - protein, 169 aa, Artificial
  His-tagged Ankyrin repeat protein + IPP
MRGSHHHHHHGSDLGKKLLEAARAGQDDEVRILMANGADVNATDNDGYTPLHLAASNGHLEIVEVLLKNGADVNASDLTGITPLHLAAATGHLEIVEVLLKHGADVNAIPPYDNDGHTPLHLAAKYGHLEIVEVLLKHGADVNAQDKFGKTAFDISIDNGNEDLAEILQ

SEQ ID NO: 4 - DNA, 510 bp, Artificial
  His-tagged Ankyrin repeat protein + IPP
atgagaggatcgcatcaccatcaccatcacggatccgacctgggtaagaaactgctggaagctgctcgtgctggtcaggacgacgaagttcgtatcctgatggctaacggtgctgacgttaacgctactgacaatgatggttatactccgctgcacctggctgcttctaatggtcacctggaaatcgttgaagttctgctgaagaacggtgctgacgttaacgcttctgaccttactggtattactccgctgcacctggctgctgctactggtcacctggaaatcgttgaagttctgctgaagcacggtgctgacgttaacgctatcccgccgtatgacaatgatggtcatactccgctgcacctggctgctaagtatggtcacctggaaatcgttgaagttctgctgaagcacggtgctgacgttaacgctcaggacaaattcggtaagaccgctttcgacatctccatcgacaacggtaacgaggacctggctgaaatcctgcaataa

SEQ ID NO: 5 - DNA, 522 bp, Artificial
  PCR fragment
ctagctagcaagtccgacctgggtaagaaactgctggaagctgctcgtgctggtcaggacgacgaagttcgtatcctgatggctaacggtgctgacgttaacgctactgacaatgatggttatactccgctgcacctggctgcttctaatggtcacctggaaatcgttgaagttctgctgaagaacggtgctgacgttaacgcttctgaccttactggtattactccgctgcacctggctgctgctactggtcacctggaaatcgttgaagttctgctgaagcacggtgctgacgttaacgctatcccgccgtatgacaatgatggtcatactccgctgcacctggctgctaagtatggtcacctggaaatcgttgaagttctgctgaagcacggtgctgacgttaacgctcaggacaaattcggtaagaccgctttcgacatctccatcgacaacggtaacgaggacctggctgaaatcctgcaagactacaaggatgatgatgacaagtaaaggcgcgcc

SEQ ID NO: 6 - DNA, 1584 bp, Artificial
  Glucoamylase fragment
ttaattaacaccgtcaaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactacttctacacctggactcgcgactctggtctcgtcctcaagaccctcgtcgatctcttccgaaatggagataccagtctcctctccaccattgagaactacatctccgcccaggcaattgtccagggtatcagtaacccctctggtgatctgtccagcggcgctggtctcggtgaacccaagttcaatgtcgatgagactgcctacactggttcttggggacggccgcagcgagatggtccggctctgagagcaactgctatgatcggcttcgggcaatggctgcttgacaatggctacaccagcaccgcaacggacattgtttggcccctcgttaggaacgacctgtcgtatgtggctcaatactggaaccagacaggatatgatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggtctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacctctgccagcagcgtgcccggcacctgtgcggccacatctgccattggtacctacagcagtgtgactgtcacctcgtggccgagtatcgtggctactggcggcaccactacgacggctacccccactggatccggcagcgtgacctcgaccagcaagaccaccgcgactgctagc

SEQ ID NO: 7 - protein, 689 aa, Artificial
  FLAG-tagged chimeric Glucoamylase-Ankyrin repeat protein containing IPP
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKSDLGKKLLEAARAGQDDEVRILMANGADVNATDNDGYTPLHLAASNGHLEIVEVLLKNGADVNASDLTGITPLHLAAATGHLEIVEVLLKHGADVNAIPPYDNDGHTPLHLAAKYGHLEIVEVLLKHGADVNAQDKFGKTAFDISIDNGNEDLAEILQDYKDDDDK

SEQ ID NO: 8 - DNA, 2070 bp, Artificial
  DNA encoding FLAG-tagged chimeric Glucoamylase-Ankyrin repeat protein containing IPP
atgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactacttctacacctggactcgcgactctggtctcgtcctcaagaccctcgtcgatctcttccgaaatggagataccagtctcctctccaccattgagaactacatctccgcccaggcaattgtccagggtatcagtaacccctctggtgatctgtccagcggcgctggtctcggtgaacccaagttcaatgtcgatgagactgcctacactggttcttggggacggccgcagcgagatggtccggctctgagagcaactgctatgatcggcttcgggcaatggctgcttgacaatggctacaccagcaccgcaacggacattgtttggcccctcgttaggaacgacctgtcgtatgtggctcaatactggaaccagacaggatatgatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggtctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacctctgccagcagcgtgcccggcacctgtgcggccacatctgccattggtacctacagcagtgtgactgtcacctcgtggccgagtatcgtggctactggcggcaccactacgacggctacccccactggatccggcagcgtgacctcgaccagcaagaccaccgcgactgctagcaagtccgacctgggtaagaaactgctggaagctgctcgtgctggtcaggacgacgaagttcgtatcctgatggctaacggtgctgacgttaacgctactgacaatgatggttatactccgctgcacctggctgcttctaatggtcacctggaaatcgttgaagttctgctgaagaacggtgctgacgttaacgcttctgaccttactggtattactccgctgcacctggctgctgctactggtcacctggaaatcgttgaagttctgctgaagcacggtgctgacgttaacgctatcccgccgtatgacaatgatggtcatactccgctgcacctggctgctaagtatggtcacctggaaatcgttgaagttctgctgaagcacggtgctgacgttaacgctcaggacaaattcggtaagaccgctttcgacatctccatcgacaacggtaacgaggacctggctgaaatcctgcaagactacaaggatgatgatgacaagtaa

SEQ ID NO: 9 - DNA, 34 bp, Artificial
  PCR primer
ctagctagcaagtccgacctgggtaagaaactgc

SEQ ID NO: 10 - DNA, 58 bp, Artificial
  PCR primer
ggcgcgcctttacttgtcatcatcatccttgtagtcttgcaggatttcagccaggtcc

SEQ ID NO: 11 - protein, 689 aa, Artificial
  FLAG-tagged designed chimeric ankyrin repeat protein containing IPP
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKSDLGEKLLEAARAGQDDEVRALMANGADVNARDSDGRTPLHLAAENGHLEIVELLLKHGADPNARDSSGETPLHLAAENGHLEIVKLLLEHGADVNAIPPKDNEGRTPLHLAAENGHLEIVELLLKHGADPNARDKFGKTAFDESRDNGNEDLAKILQDYKDDDDK

SEQ ID NO: 12 - DNA, 2070 bp, Artificial
  DNA encodingFLAG-tagged designed chimeric ankyrin repeat protein containing IPP
atgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactacttctacacctggactcgcgactctggtctcgtcctcaagaccctcgtcgatctcttccgaaatggagataccagtctcctctccaccattgagaactacatctccgcccaggcaattgtccagggtatcagtaacccctctggtgatctgtccagcggcgctggtctcggtgaacccaagttcaatgtcgatgagactgcctacactggttcttggggacggccgcagcgagatggtccggctctgagagcaactgctatgatcggcttcgggcaatggctgcttgacaatggctacaccagcaccgcaacggacattgtttggcccctcgttaggaacgacctgtcgtatgtggctcaatactggaaccagacaggatatgatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggtctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacctctgccagcagcgtgcccggcacctgtgcggccacatctgccattggtacctacagcagtgtgactgtcacctcgtggccgagtatcgtggctactggcggcaccactacgacggctacccccactggatccggcagcgtgacctcgaccagcaagaccaccgcgactgctagcaagtccgaccttggtgaaaagctccttgaggctgctcgcgctggtcaggatgacgaggtccgcgccttgatggccaacggtgccgatgtcaacgcccgtgactcggatggccgcacccccttgcacctggctgctgagaacggccacctggaaattgttgagctcctcctcaagcacggtgccgaccccaacgcccgtgacagctctggtgagactcctctgcacctggctgccgagaacggccacctcgagatcgtcaagctcctcctcgaacacggtgccgatgtcaacgccatccctcccaaggacaacgagggtcgcacccccttgcacctggctgctgagaacggccacctggagatcgttgagcttctcctcaagcacggtgccgaccccaacgcccgtgacaagttcggcaagaccgctttcgacgagtcccgtgacaacggcaacgaagatcttgctaagatcctgcaggactacaaggatgacgatgacaagtaa

SEQ ID NO: 13 - protein, 701 aa, Artificial
  FLAG-tagged designed chimeric ankyrin repeat protein containing IPP, KexB site and His-tag
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKRMRGSHHHHHHGSDLGEKLLEAARAGQDDEVRALMANGADVNARDSDGRTPLHLAAENGHLEIVELLLKHGADPNARDSSGETPLHLAAENGHLEIVKLLLEHGADVNAIPPKDNEGRTPLHLAAENGHLEIVELLLKHGADPNARDKFGKTAFDESRDNGNEDLAKILQDYKDDDDK

SEQ ID NO: 14 - DNA, 2106 bp, Artificial
  Designed chimeric ankyrin repeat protein containing IPP, KexB site and His-tag N.A.
atgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactacttctacacctggactcgcgactctggtctcgtcctcaagaccctcgtcgatctcttccgaaatggagataccagtctcctctccaccattgagaactacatctccgcccaggcaattgtccagggtatcagtaacccctctggtgatctgtccagcggcgctggtctcggtgaacccaagttcaatgtcgatgagactgcctacactggttcttggggacggccgcagcgagatggtccggctctgagagcaactgctatgatcggcttcgggcaatggctgcttgacaatggctacaccagcaccgcaacggacattgtttggcccctcgttaggaacgacctgtcgtatgtggctcaatactggaaccagacaggatatgatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggtctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacctctgccagcagcgtgcccggcacctgtgcggccacatctgccattggtacctacagcagtgtgactgtcacctcgtggccgagtatcgtggctactggcggcaccactacgacggctacccccactggatccggcagcgtgacctcgaccagcaagaccaccgcgactgctagcaagcgcatgcgtggatcccaccaccaccaccaccacggatccgaccttggtgaaaagctccttgaggctgctcgcgctggtcaggatgacgaggtccgcgccttgatggccaacggtgccgatgtcaacgcccgtgactcggatggccgcacccccttgcacctggctgctgagaacggccacctggaaattgttgagctcctcctcaagcacggtgccgaccccaacgcccgtgacagctctggtgagactcctctgcacctggctgccgagaacggccacctcgagatcgtcaagctcctcctcgaacacggtgccgatgtcaacgccatccctcccaaggacaacgagggtcgcacccccttgcacctggctgctgagaacggccacctggagatcgttgagcttctcctcaagcacggtgccgaccccaacgcccgtgacaagttcggcaagaccgctttcgacgagtcccgtgacaacggcaacgaagatcttgctaagatcctgcaggactacaaggatgacgatgacaagtaa